(19) United States
(12) Patent Application Publication
(10) Pub. No.: US 2015/0285823 A1
(43) Pub. Date: Oct. 8, 2015

(54) COMPOSITIONS AND METHODS RELATED TO DORMANT SENESCENCE-PRONE CELLS (DSPC)

(71) Applicant: Health Research, Inc., Buffalo, NY (US)

(72) Inventors: Andrei Gudkov, East Aurora, NY (US); Katerina Leonova, East Aurora, NY (US)

(21) Appl. No.: 14/680,540

(22) Filed: Apr. 7, 2015

Related U.S. Application Data
(60) Provisional application No. 61/976,213, filed on Apr. 7, 2014.

(51) Int. Cl. G01N 33/68

(52) U.S. Cl. CPC G01N 33/6893; G01N 2440/14; G01N 2333/4703; G01N 2333/938; G01N 2333/5412; G01N 2333/5421; G01N 2333/535

(57) ABSTRACT

Provided is the discovery that dormant senescence prone cells (DSPCs) record an organism's exposure to genotoxic stress over the lifetime of the organism. The disclosure includes identifying DSPCs, using the amount of DSPCs to determine genotoxic dosage/dosimetry, and using these determinations in treatment and therapeutic approaches.

### CROSS REFERENCE TO RELATED APPLICATIONS

This application claims priority to U.S. Provisional patent application No. 61/976,213, filed Apr. 7, 2014, the disclosure of which is incorporated herein by reference.

### FIELD OF THE INVENTION

This disclosure relates generally to compositions and methods for diagnosis, prophylaxis, therapy and other approaches related to aging and irreversibly arrested senescent cells.

### BACKGROUND OF THE INVENTION

During their life time, living organisms frequently experience genotoxic stresses resulting in DNA damage and requiring emergency physiological responses to mitigate the resulting risks. For example, DNA damage can occur as a result of exposure to physical (i.e, UV and ionizing radiation), chemical (natural and synthetic DNA damaging compounds) and biological (pathogens such as viruses, transposable genetic elements, DNA replication errors, activation of dominant oncogenes) and can reflect environmental conditions (i.e., level of oxidative stress) or special circumstances such as, for example, nuclear accidents or cancer treatment with radiation and/or chemotherapeutic agents.

Development of assays which would allow one to quantitatively estimate the scale of genotoxicity experienced by a given organism (also referred to in the art as iodosimetry) is important for the prognosis of occurrence and severity of pathologies resulting from the exposure to genotoxic conditions and for planning medical intervention to prevent or treat such pathologies. This need is applicable not only to situations of acute DNA damage but also to such universally developed pathologies as aging. At present, there are no objective biological assays enabling one to estimate biological age of the organism as a function of accumulated genotoxicity. These needs are ongoing and well recognized in the art (see, for example Swartz et al, A critical assessment of biodosimetry methods for large-scale incidents. Health Phys. 2010 February; 98(2):95-108), and numerous groups are engaged in development of various approaches to biodosimetry, all of which stem from knowledge about the mechanisms of organismal response to genotoxic exposures. However, there are no reliable approaches available that would enable accurate analysis of the cumulative DNA damage an organism has experienced. Thus, there is an ongoing and unmet need for improved methods for biodosimetry, and for use of such methods in diagnostics and therapeutic approaches. The present disclosure meets these and other needs.

### SUMMARY

The present disclosure is based at least in part on the presently disclosed discoveries which show that genotoxic conditions experienced by mammalian organisms (e.g., exposure to UV or ionizing radiation, treatment with chemotherapeutic drugs and other oxidative stresses) and resulting in DNA damage are not repaired by DNA repair systems in mesenchymal cells, but remain unrecognized and can stay unrepaired for extensive time periods. Without intending to be constrained by any particular theory, it is considered that the unrepaired DNA can persist for the entire life of the organism. Further, and again, without wishing to be bound by theory, it is believed that triggering a DNA damage response in such mesenchymal cells occurs when they are subjected to stimuli that typically promotes cell division—such after they are plated in tissue culture, or at the sites of tissue wounding. An attempt to enter the cell cycle results in conversion of such cells, in a p53-dependent manner, into physiological state of irreversible growth arrest known as cellular senescence. Hence, accumulation of senescent cells in vivo is a two-step process that includes (i) initiation (appearance of dormant senescence-prone cells or DSPCs) and (ii) a promotion step (conversion into senescence by proliferation-inducing stimuli, or stimuli that would typically induce proliferation). These observations now for the first time reveal the existence of DSPCs as a natural memory mechanism that records genotoxic events that take place within the organism during its life time. The present disclosure provides that the proportion of such cells among mesenchymal cells in tissues is a quantitative measure of cumulative genotoxicity experienced by a given organism, and therefore can be used as an approach to biodosimetry. Methods of detection of such cells can involve the use of the biomarkers disclosed herein as specifically expressed by DSPC and/or quantitation of the proportion of senescent cells in mesenchymal cell populations following promoting proliferation (promotion step). DSPC-based biodosimetry can be applied to various areas of medicine, including determination of severity of damage following exposure to genotoxic treatments (nuclear disasters, cancer treatment side effects) and estimation of physiological age as a function of cumulative DNA damage, and for use in treatment decisions, and for targeting DSPCs in individuals in need thereof.

Thus, it will be recognized from the foregoing that, in general, the present disclosure provides compositions and methods for estimating a prior dose of genotoxic exposure of an organism, or an organ, or a tissue, or a cell population. As used herein, the terms “genotoxicity” and “genotoxic” refer to the effects of exogenous stimuli, events and/or agents that damage DNA. In embodiments, the present disclosure includes approaches that can serve as a surrogate for determining a prior genotoxic exposure, and the amount of such exposure. In embodiments, the genotoxic exposure comprises exposure to radiation, whether or not the exposure was intentional, such as a result of a medical imaging procedure, or accidental, such as inadvertent proximity to a source of radiation without adequate protection. Exposure to ionizing radiation and ultraviolet radiation are included. Thus, in embodiments, the disclosure encompasses determining biodosimetry of an organism. In embodiments, the genotoxic exposure can include treatment or other exposure of an individual with chemical agents that adversely modify nucleic acids, and in particular modify DNA such that the DNA is subjected to single stranded nicking events, or double stranded breaks, or other modification of nucleic acids, including cross-linking or other covalent modifications.

It will be apparent from the foregoing to those skilled in the art that in one aspect, the disclosure provides a method for determining an amount of dormant senescence prone cells in an individual. The method generally comprises: a) obtaining a biological sample comprising mesenchymal cells from a human individual or non-human animal; b) placing the biological sample under conditions which promote cell proliferation, and subsequently measuring indicia of DNA damage response in the mesenchymal cells to obtain a measurement of the amount of dormant senescence prone cells in the biological sample, wherein the DNA damage response is in the dormant senescent prone cells, and wherein the amount of dormant senescent prone cells is a proportion of the mesenchymal cells.

In embodiments, the indicia of DNA damage response is compared to a reference to obtain a measurement of the degree of genotoxic stress the human individual or non-human animal from which the biological sample was obtained experienced during its lifetime, but before the sample was obtained. It will also be recognized from the data presented herein that the step of promoting the cells to proliferate can comprise, for example, plating the cells in culture to provide those cells that can proliferate the opportunity to do so. However, it will also be recognized that mesenchymal cells that have sustained DNA damage and have been converted into DSPCSs do not proliferate. Instead, it is believed when DSPCs are promoted to proliferate, they attempt to enter cell cycle, but then senesce. Thus, the DSPCS do not pass through mitosis. A lack of proliferation may therefore in and of itself be indicative of DSPCs as the non-proliferating cells. Accordingly, the proportion of non-proliferating mesenchymal cells in a biological sample that has been placed in conditions which ordinarily promote proliferation in vitro may itself be indicative of the proportion of DSPCs in the sample, and thus a measure of genotoxic exposure. In the present specification, the term “promoting” proliferation means exposing cells to stimuli that would ordinarily result in proliferation, but does not necessitate proliferation when used in reference to DSPCs, which as described above, do not proliferate.

In embodiments, the genotoxic stress comprises exposure to ionizing radiation, or having been treated with one or more chemotherapeutic drugs which damage DNA, or a combination of the ionizing radiation and exposure to the chemotherapeutic drug.

In one aspect, promoting the proliferation of the mesenchymal cells is performed ex vivo using biological sample that comprises a tissue sample. In a related aspect, promoting the proliferation of the mesenchymal cells is performed after plating and culturing the mesenchymal cells in vitro.

In embodiments, the method comprises comparing a measurement of indicia of DNA damage to a suitable reference, i.e., a control. In embodiments, comparison to a reference comprises testing a first biological sample comprising mesenchymal cells obtained from the individual, and comparing indicia of DNA damage to a second biological sample comprising mesenchymal cells obtained from the individual. In embodiments, this approach comprises: a) in the first biological sample, measuring indicia of DNA damage response in the mesenchymal cells after the placing them in the conditions promoting proliferation, and allowing a period of time to pass during which proliferation takes place in cells that do not exhibit the DNA damage response; and b) in the second biological sample, measuring indicia of the DNA damage response before promotion of proliferation (pre-proliferation promotion cells). An increase in the indicia of the DNA damage response in the cells of a) relative to the indicia of DNA damage response in the pre-proliferation cells of b) indicates the biological sample comprised dormant senescent prone cells. The amount of increase in the indicia comprises a measurement of the degree of genotoxic stress the human individual or non-human animal experienced during its lifetime before the sample was obtained. As an alternative to using the second biological sample, a reference can comprise a series of cell or tissue samples of the same species subjected to a range of controlled doses of genotoxic treatments.

In certain approaches, the indicia of DNA damage that is determined according to this disclosure comprises any one or any combination of determining: phosphorylation of a histone, nuclear foci comprising 53BP1, nuclear foci comprising Rad51, phosphorylation of RPA32, or secretion of a cytokine associated with senescence-associated secretory phenotype (SASP), wherein the cytokine is selected from interleukins, such as IL6 and IL8, and Granulocyte-colony stimulating factor (GCSF). In certain embodiments, the phosphorylation of the histone or the phosphorylation of RPA32, or the nuclear foci comprising 53BP1, or RPA32, or a combination thereof, is determined using an suitable immunological assay. In embodiments, the histone that is phosphorylated and detected an H2A histone.

It will be apparent that the disclosure leads to the capability to make prognostic and diagnostic recommendations to a patient, and/or to aid in a physician's diagnosis and/or recommendations, and treatment decisions. Thus in embodiments, wherein the biological sample is determined to comprise DSPCs, and/or an amount of DSPCs greater than a suitable reference, the method further comprises recommending that the individual avoid weight gain, and/or recommending that the individual avoid exposure to ionizing radiation, and/or modifying a chemotherapeutic approach to lessen the amount or eliminate the use of chemotherapeutic agents that are known to function by damaging DNA.

In embodiments, the disclosure comprises determining that the biological sample comprises DSPCs, and further comprises determining the degree of the indicia of the DNA damage and estimating an amount of one or more DNA damaging agents received by the individual before the biological sample was obtained.

In a related aspect, the disclosure includes determining that the biological sample comprises DSPCs, and further comprises assigning a biological age to the individual, wherein the biological age is greater than the chronological age of the individual.

In one embodiment, the disclosure comprises determining that the biological sample comprises DSCPs, and/or an amount of DSCPs that is greater than a suitable reference, and further comprises administering to the individual an agent that selectively kills dormant senescent cells.

### BRIEF DESCRIPTION OF THE FIGURES

FIGS. 1A-1C: Mouse mesenchymal cells isolated from 11Gy total body irradiated in vivo C57Bl/6 mice from various tissues (lung, kidney, heart and muscle). Cells derived from untreated animals when placed in vitro proceeded to proliferate, whereas cells isolated from irradiation treated animals ceased proliferation when placed in culture. The same effect was observed when the cells were isolated and placed in culture at various time points after radiation treatment at 7 (FIG. 1A), 14 (FIG. 1B) and 28 (FIG. 1C) days.

FIG. 2. Graph showing comparison of the doubling capacity of lung mesenchymal cells isolated from radiation treated and untreated mice.

FIGS. 3A-3B. Graphs showing numbers of lung mesenchymal cells isolated 72 hours following either 0, 1, 5 or 15Gy of radiation (FIG. 3A) or cells isolated after 11Gy of TBI after 5 days or 5 months (FIG. 3B).

FIG. 4: Graph showing measurement of lung mesenchymal cell proliferation in cells isolated from radiation treated mice, as measured by EdU incorporation. Results obtained from measuring control (LF) cells and IR-treated (LFIR) cells are represented in the graph.

FIGS. 5A-5C: Assays of senescence associated markers. (FIG. 5A) Senescence associated beta-galactosidase activity measured in mesenchymal lung cells following treatment with different doses of gamma-radiation. (FIG. 5B) Western immunoblotting for phosphorylated gamma-H2AX protein. (S=senescent; P=proliferation; dox=doxorubicin). H2AX (H2A histone family member X) becomes phosphorylated under the conditions of double-stranded DNA break. (FIG. 5C) Western blot for anti-HMGB1 antibody.

FIG. 6: Images of immunohistochemistry analysis of cells treated with different DNA damaging markers that detect various types of damage, including double strand and single strand breaks.

FIG. 7: Graphs showing measurements of markers of senescence-associated secretory phenotype (SASP).

FIGS. 8A-8B: Graphs showing cell cycle distribution (FIG. 8A) and EdU incorporation (FIG. 8B).

FIGS. 9A-9D: Graphs providing analysis of whether senescence observed in irradiation treated cells is p53 dependent. (FIG. 9A) Cell doubling determined by crystal violet over 11 days. (FIG. 9B) Staining of IR-treated and untreated cells with beta-galactosidase. (FIG. 9C) Senescence associated secretory phenotype assayed in p53 wild-type and null cells isolated from IR-treated and untreated lung tissue. (FIG. 9D) Graph showing EdU incorporation in mesenchymal lung cells isolated from p53-null radiation treated and untreated mice.

FIG. 10: Venn diagram represents the number of genes that were upregulated in mouse lung fibroblasts isolated after 5 days post IR or 5 months post IR in comparison to untreated proliferating control.

FIG. 11: Graph of ILLUMINA microarray-based analysis of gene expression in mouse lung derived fibroblasts in group that received in vivo gamma-radiation and were sacrificed either 5 days or 5 months after treatment.

FIG. 12: Images of various tissues of untreated and IR treated animals.

FIG. 13: Images of EdU incorporation in small intestine of intact (untreated) and IR-treated animals.

FIG. 14: Venn diagram of microarray analysis performed on mouse lung tissue that varied in radiation treatment time and age.

FIG. 15: Graph showing genes upregulated in mouse lung tissue obtained from both irradiated and naturally aged mice.

FIG. 16. Graphical summary of a frailty index (FI).

FIG. 17. Image and graphs obtained from analysis of C57Bl/6 mice, with and without radiation (IR) maintained either on a normal mouse diet (11% Fat) or a high-fat mouse diet (58%) (HF).

FIG. 18. Graph (middle), Western blot (inset) and images (bottom panel) from analysis of primary mouse lung fibroblasts from C57Bl/6 mice 72 hours after various doses of total body of irradiation (0, 1, 5, 11, and 15Gy).

FIG. 19. Characterizations of DNA damage repair in mouse lung fibroblasts isolated from irradiated versus non-irradiated mice showing pulsed gel electrophoresis cartoon and gel, graph (middle) and Western blot (right panel).

FIG. 20. Cartoon and images generated from data obtained in demonstrating that DSPCs that placed under conditions that induce proliferation become senescent cells.

FIG. 21. Images demonstrating that the DSPC microenvironment greatly enhances growth of experimental metastases of melanoma in lungs.

### DESCRIPTION OF THE INVENTION

The present disclosure provides compositions and methods for use in biodosimetry related approaches to improving health. As discussed above, current approaches to biodosimetry are based on quantitation of the degree of remaining DNA damage (i.e., chromosomal aberrations, indications of physical breaks in DNA), detection of biochemical parameters of ongoing DNA damage response (expression and assembly of proteins DNA damage recognition and repair in nuclear plaques, proteins involved in cell cycle arrest, phosphorylation of specific components of chromatin, etc.) or their metabolic consequences. All of these parameters (with partial exception of chromosomal rearrangements) are transient and do not provide useful information about past genotoxic events. Thus, the present disclosure describes and provides methods of manipulating and using what can be considered an equivalent of long lasting memory, which accumulates over preexisting “records” of genotoxic events as newly occurred ones in the form of increasing the proportion of DSPC and density of DNA damage in them. The present disclosures provides in various embodiments compositions and methods that reveal this memory by “development” of hidden unrecognized DNA damage which is achievable by exposing cells to proliferation inducing conditions.

Thus, in general, the present disclosure is based at least in part on the discovery of a physiological outcome of mammalian cells in response to genotoxic conditions, which has heretofore been unreported. In particular, we found that cells of mesenchymal differentiation, after they experience DNA damaging treatment in vivo (i.e., inside tissues), do not exhibit known or expected physiological responses. For example, they neither activate DNA repair, nor undergo apoptosis or acquire a senescent phenotype. They remain physiologically active and can persist with damaged DNA for the entire life of the organism or as long as they are not provoked to enter the cell cycle. However, if subjected to changes in environmental conditions (plating in culture, tissue wounding, etc.) they are promoted to proliferate, and they activate “classical” DNA damage responses, followed by p53-dependent conversion into senescence. The proportion of such cells depends on the dose of genotoxic treatment, can reach close to 100% of the entire mesenchymal cell population, does not change with time and therefore can be used, in combination with the scale of DNA damage in individual cells, as a universal measure of cumulative genotoxicity experienced by the organism. Thus, some advantages of the approaches described in this disclosure include but are not necessarily limited to our discovery that the proportion of accumulated DSPCs is a stable parameter and does not depend on time that passed after exposure to genotoxic stress.

The approaches of this disclosure are in embodiments a cumulative assessment of the overall degree of DNA damage regardless of its nature, origin and time since the damage occurred. This disclosure accordingly enables determining DNA damage in chronic and acute conditions of exposure to genotoxic stresses.

The aging process involves systemic accumulation of irreversibly arrested senescent cells that are believed to contribute to development of age-related diseases by poisoning organism with bioactive secreted factors (senescence-associated secretory phenotype or SASP). Since establishment of senescence is a response of mesenchymal cells to genotoxic stresses in vitro, one would expect that exposure of mammalian organism to severe genotoxic stress in vivo should result in accumulation of senescent cells and accelerated aging. Surprisingly, however, mice that received high doses (7-13 Gy) of total body irradiation and rescued from radiation-induced lethality by bone marrow transplantation manifest only a limited subset of aging traits and do not show a substantial decrease in their natural life span. Lack of massive appearance of senescent cells in vivo following lethal irradiation strikingly contrasted with complete inability of mesenchymal cells from organs of irradiated mice to proliferate in tissue culture and their 100% conversion to a complete senescence phenotype. Importantly, the commitment of mesenchymal cells from tissues of irradiated mice to senescence in vitro remained unchanged during the entire mouse life. Conversion to senescence is preceded with cell attempting to resume the cell cycle and activation of DNA damage response, which was not activated in these cells in vivo following TBI. These observations fit the following model, which is intended to illustrate but not limit embodiments of this disclosure. Systemic genotoxic stress creates conditions enabling accelerated aging by initiating massive accumulation of cells predisposed to senescence, but not yet displaying a fully developed senescent phenotype (dormant senescence-prone cells or DSPCs). Accelerated aging occurs when DSPCs are promoted to a senescent state, as a result of exposure to proliferation inducing conditions that reveal their senescence-prone capabilities, leading to formation of a massive pool of senescent cells. Initiated DSPCa can stay in the organism indefinitely, thus memorizing individual life history of genotoxicity and determining the speed of aging development under conditions favoring the promotion of dormant senescence-prone cells to bone fide senescence state. Potential implications of these findings to biodosimetry of genotoxicity and to prophylaxis of accelerated aging in people subjected to genotoxic stresses are aspects of this disclosure.

In embodiments the disclosure includes use of DSPC for diagnostic purposes (biodosimetry of genotoxic stresses), such as for diagnosing and/or aiding in a physician's diagnosis of a condition that is associated with DSPC. In embodiments the disclosure includes method to detect (i.e., diagnose) the amount of accumulated genotoxic stress in mammalian organism. Genotoxic stress includes but is not necessarily limited to radiation, effects of chemotherapeutic agents, natural and synthetic poisons, and other types of oxidative stresses.

In various embodiments the disclosure includes methods of determining the biological age of an organism, methods for the quantitative estimation of the dose of radiation received by the organism, and methods for detection of DNA damage acquired after chemotherapeutic treatment.

In another aspect the disclosure included prophylaxis and/or therapy of pathologies associated with DSPC. In embodiments this aspect includes methods of prophylaxis of aging and/or age-related diseases by reducing and/or eradication of DSPCs. Alternatively, such approaches can include activation of DNA repair and reversion of DSPC into normal state.

In one aspect of this disclosure, DSPC can are provided as research tools that useful for multiple applications, including but not necessarily for the screening, selection, design and testing for pharmacological agents that can cause a reduction or eradication of the DSPCs. Thus, in embodiments, the disclosure includes methods for screening of a library of pharmacological compounds aimed to selectively kill DSPC cells, methods for screening of a library of pharmacological compounds aimed to isolate compounds responsible for the induction of DNA repair in DSPC, and methods of modeling natural and accelerated aging by combining conditions that lead to massive accumulation of DSPC in vivo (e.g., total body irradiation, chemotherapy with DNA damaging agents, etc.) followed by applying conditions promoting massive conversion of DSPC into senescent state (e.g., high fat diet, use of growth stimulating hormones, wounding, etc.).

The present disclosure provides representative demonstrations of properties of DSPCs and embodiments which comprise methods of differential detection of DSPC based on the identification of differential expression of one or more genes in proliferating versus senescent cells such as those listed in Table 1 and 2, and methods for differential detection of DSPC based on the identification of differential expression of one or more genes in the tissues of young, irradiated and old tissues as listed in Table 3, 4 and 5. The markers described in these Tables are described by nomenclature used in the art (i.e., in the column labeled “Target ID”), and the skilled artisan can readily identify their polynucleotide and amino acid sequences, as the case may be, given the benefit of this disclosure.

Thus, it will be apparent from the foregoing that the present disclosure includes various aspects which involve characterization of DSPCs, such as in a whole subject or in suitable biological samples obtained from a subject, screening of a plurality of test agents to identify test agents as candidates for modulating one or more conditions correlated with DSPCs, and for use in reducing or eradicating DSPCs from a subject, methods for prophylaxes and/or therapy of such conditions by administering to a subject a pharmaceutical composition in an amount effective to reduce or eradicate DSPCs from a subject, and a host of research tools that relate to use of DSPCs in a wide range of research applications.

In embodiments the disclosure comprises testing for the presence, absence, or amount, of any one or any combination of the markers described herein. In embodiments, the disclosure comprises testing for the presence, absence, or amount, of any one or any combination of the markers in Tables 1 and 2, and/or testing for the presence, absence, or amount, of any one or any combination of the markers Tables 3, 4 and 5. All combinations of the markers are included. The disclosure also includes excluding any one, or any combination of the markers. Thus, in embodiments, the disclosure includes testing for one or more markers, wherein the one or more markers can be present with other markers, or can be the only DSPC markers tested, and wherein in certain embodiments the only DSPC markers tested can comprise or consist of any one or any combination of the markers described herein.

In order to qualitatively or quantitatively assess the markers, comparisons can be made to any suitable control, including but not necessarily limited to positive controls, negative controls, standardized controls, an area under a curve, or any other suitable representation of a standard with which the presence and/or amount of the DSPC markers can be compared. In embodiments, a positive control comprises cells which have not undergone DNA damage, and/or are not irreversibly arrested senescent cells, and/or are cells or a sample from a subject which have a known chronological or biological age, or have undergone a known or controlled number of divisions, or, for example, have not been exposed to radiation or a chemotherapeutic agent. In embodiments, markers from proliferating cells are compared to senescent cells, and/or expression of the markers in tissues of young, irradiated and old tissues are compared. In embodiments, the reference comprises a plurality of cells or tissue samples of the same species that have been subjected to a range of controlled doses of genotoxic treatment, and an average or other value based on measuring indicia of DNA damage in such samples is used.

In embodiments, testing the sample comprises measuring a polynucleotide or a protein that is a marker disclosed herein. In embodiments, testing the sample comprises forming and detecting a non-naturally occurring complex of a marker and a specific binding partner, such as a detectably labeled oligonucleotide probe or an antibody. In embodiments, testing the sample comprises detecting and/or quantitating nucleic acids using a microarray or “chip” approach. In embodiments the testing comprises amplifying nucleic acids using a composition comprising primers and a recombinant DNA polymerase, such as in a PCR reaction.

In embodiments, testing the samples comprises generating a Frailty Index as further described herein, such as a Frailty Index (FI) for a subject who is tested for DSPC markers.

In embodiments, articles of manufacture are provided. The articles can contain printed material and packaging. The printed material can include an indication that the contents of the packaging are intended for prophylaxis and or therapy of any condition associated with any of the DSPC marker(s) disclosed herein. In other embodiments, the printed material provides an indication that the contents of the packaging are for testing for DSPC markers, and/or for making a diagnosis of a condition associates with the DSPC markers, or for aiding a physician in making such a diagnosis.

The disclosure includes fixing in a tangible medium of expression the results of testing for the DSCPC markers, such as in an electronic file. The disclosure includes transferring such medium to a health care provider. The disclosure includes making treatment or other behavioral recommendations, or providing a prognosis, based on the testing of the markers.

The disclosure also comprises administering to an individual an effective amount of an agent that can selectively target DSCPCs, thereby reducing or eliminating them from the subject and as a consequence mitigating conditions associated with the presence of the DSCPCs. The disclosure also includes administering to an individual an effective amount of an agent that can inhibit the formation of DSCPCs.

It will accordingly be apparent from the foregoing that the present disclosure generally comprises: a) obtaining a biological sample comprising mesenchymal cells from a human individual or non-human animal; b) placing the biological sample under conditions which promote cell proliferation, and subsequently measuring indicia of DNA damage response in the mesenchymal cells to obtain a measurement of the amount of dormant senescence prone cells in the biological sample, wherein the DNA damage response is in the dormant senescent prone cells, and wherein the amount of dormant senescent prone cells is a proportion of the mesenchymal cells. In embodiments, the indicia of DNA damage response is compared to a reference to obtain a measurement of the degree of genotoxic stress the human individual or non-human animal from which the biological sample was obtained experienced during its lifetime, but before the sample was obtained.

In embodiments, the genotoxic stress comprises exposure to ionizing radiation, or having been treated with a chemotherapeutic drug which damages DNA, or a combination of the ionizing radiation and exposure to the chemotherapeutic drug.

In one aspect, promoting the proliferation of the mesenchymal cells is performed ex vivo using biological sample that comprises a tissue sample. In a related aspect, promoting the proliferation of the mesenchymal cells is performed after plating and culturing the mesenchymal cells in vitro. In embodiments, the method comprises comparing a measurement of indicia of DNA damage to a suitable reference, i.e., a control. In embodiments, comparison to a reference comprises testing a first biological sample comprising mesenchymal cells obtained from the individual, and comparing indicia of DNA damages to a second biological sample comprising mesenchymal cells obtained from the individual. This approach generally comprises use of a first biological sample obtained from the individual, and as a reference a second biological sample comprising mesenchymal cells from the individual, the method comprising: a) in the first biological sample, measuring indicia of DNA damage response in the mesenchymal cells after placing them in the conditions promoting proliferation, and allowing a period of time to pass during which proliferation takes place in cells that do not exhibit the DNA damage response; and b) in the second biological sample, measuring indicia of the DNA damage response before promotion of proliferation (pre-proliferation promotion cells); wherein an increase in the indicia of the DNA damage response in the cells of a) relative to the indicia of DNA damage response in the pre-proliferation cells indicates the biological sample comprised dormant senescent prone cells. Thus, the amount of increase in the indicia comprises a measurement of the degree of genotoxic stress the human individual or non-human animal experienced during its lifetime before the sample was obtained. Accordingly, the present disclosure reveals that an increase in the amount of the indicia of DNA damage in the cells given time to proliferate (but do not proliferate in the case of DSPCs) relative to the pre-proliferation cells comprises a measurement of the degree of genotoxic stress the human individual or non-human animal experienced during its lifetime before the sample were obtained. In embodiments, the first and second biological samples are obtained from dividing a single sample into first and second biological samples. With respect to the period of time that passes during which proliferation takes place, such parameters are well known in the art. In embodiments, this time period comprises or consists of between 1 and 168 hours, including all integers and ranges of integers there between. In embodiments, the time period is not more than 72 hours, or not more than 24 hours, or not more than 12 hours. In embodiments, the indicia of the DNA damage response in the pre-proliferation promotion cells is determined before the cells attach to a culture medium or culture substrate.

In certain approaches, the indicia of DNA damage that is determined according to this disclosure comprises any one or any combination of determining: phosphorylation of a histone, nuclear foci comprising 53BP1, nuclear foci comprising Rad51, phosphorylation of RPA32, or secretion of a cytokine associated with senescence-associated secretory phenotype (SASP), wherein the cytokine is selected from interleukins, such as IL6 and IL8, and Granulocyte-colony stimulating factor (GCSF). In certain embodiments, the phosphorylation of the histone or the phosphorylation of RPA32, or the nuclear foci comprising 53BP1, or RPA32, or a combination thereof, is determined using an suitable immunological assay. In embodiments, the histone that is phosphorylated and detected an H2A histone.

It will be apparent that the disclosure leads to the capability to make prognostic and diagnostic recommendations to a patient, and/or to aid in a physician's diagnosis and/or recommendations, and treatment decisions. Thus in embodiments, wherein the biological sample is determined to comprise DSPCs, and/or an amount of DSPCs greater than a suitable reference, the method further comprises recommending that the individual avoid weight gain, and/or recommending that the individual avoid exposure to ionizing radiation, and/or modifying a chemotherapeutic approach to lessen the amount or eliminate the use of chemotherapeutic agents that are known to function by damaging DNA.

In embodiments, the disclosure comprises determining that the biological sample comprises DSPCs, and further comprises determining the degree of the indicia of the DNA damage and estimating an amount of one or more DNA damaging agents received by the individual before the biological sample was obtained.

In a related aspect, the disclosure includes determining that the biological sample comprises DSPCs, and further comprises assigning a biological age to the individual, wherein the biological age is greater than the chronological age of the individual.

In one embodiment, the disclosure comprises determining that the biological sample comprises DSCPs, and/or an amount of DSCPs that is greater than a suitable reference, and further comprises administering to the individual an agent that selectively kills dormant senescent cells.

The following specific examples are provided to illustrate the invention, but are not intended to be limiting in any way.

### EXAMPLE 1

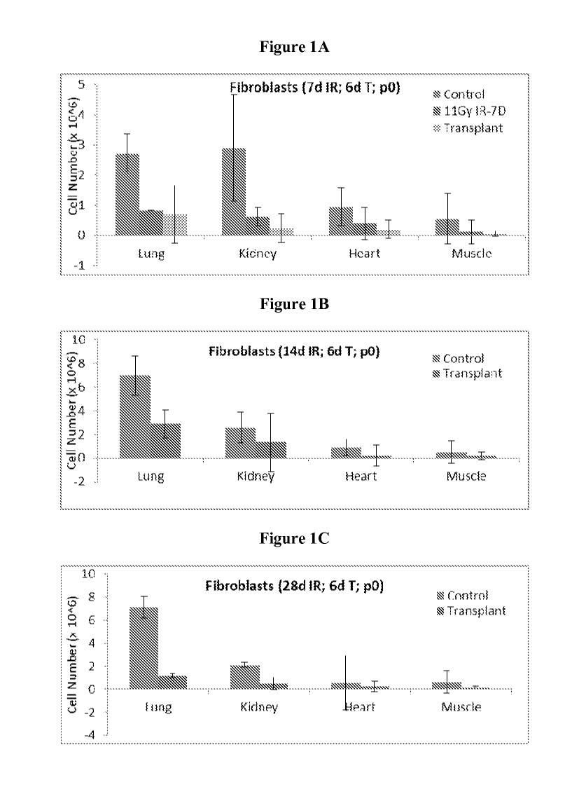

This Example demonstrates that mesenchymal cells isolated from irradiation treated mice fail to proliferate in culture, and that this effect can be detected weeks after radiation. In this regard, FIGS. 1A-1C summarize in bar graphs data obtained from analysis of mouse mesenchymal cells isolated from 11Gy total body irradiated in vivo C57Bl/6 mice from various tissues (lung, kidney, heart and muscle). The cells were isolated using 2 mg/ml of Dispase II (Roche) for 90 min digestion. Cells derived from untreated animals when placed in vitro proceeded to proliferate, whereas cells isolated from irradiation treated animals ceased proliferation when placed in culture. Moreover, the same effect was observed when the cells were isolated and placed in culture at various time points after radiation treatment at 7 (FIG. 1A), 14 (FIG. 1B) and 28 (FIG. 1C) days. Bone marrow transplantation was used to rescue the mice from lethal 11Gy irradiation. Mesenchymal cells isolated from irradiation treated mice fail to proliferate in culture. This effect was detected weeks after radiation, thus indicating that mesenchymal cells form a memory of acquiring DNA damage.

### EXAMPLE 2

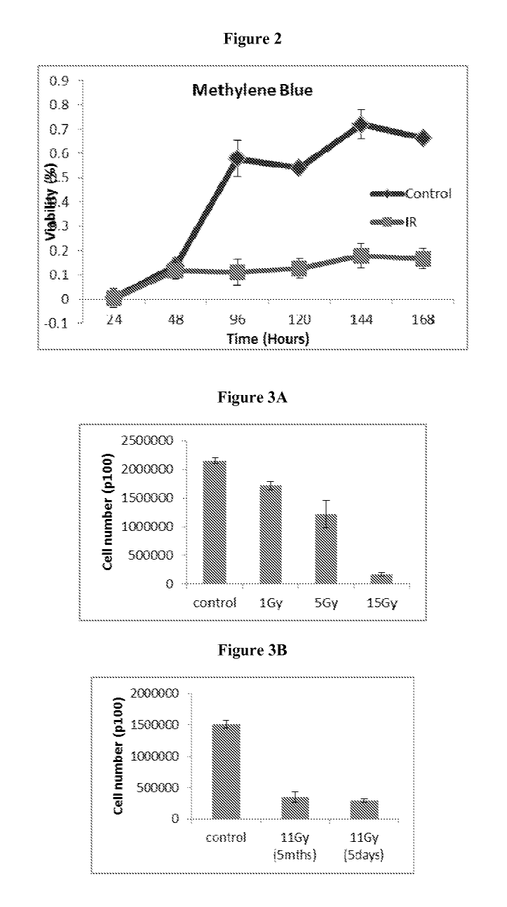

This Example demonstrates the effects on cell proliferation induced by radiation. In this regard, as shown in FIG. 2, to compare the doubling capacity of lung mesenchymal cells isolated from radiation treated and untreated mice, the viability was assayed by methylene blue at various time points after plating. One thousand cells were plated per well in 96 well-plate in triplicate; cells were fixed and stained using methylene blue. The experiment lasted 168 hours and we determined that cells isolated from untreated animals continue proliferation, whereas cells isolated from radiation treated animals do not.

### EXAMPLE 3

This Example provides an analysis of the influence of time elapsed after radiation, versus the effect of just the dosage of radiation itself. The results are presented in FIGS. 3A-3B. To determine whether it is the time after radiation or whether the dose of the radiation is critical for the termination of cell division, lung mesenchymal cells were isolated 72 hours following either 0, 1, 5 or 15Gy of radiation (FIG. 3A) or cells were isolated after 11Gy of TBI after 5 days or 5 months (FIG. 3B). The experiment showed that the dose of the radiation is more critical than the length of time passed after the IR-treatment.

### EXAMPLE 4

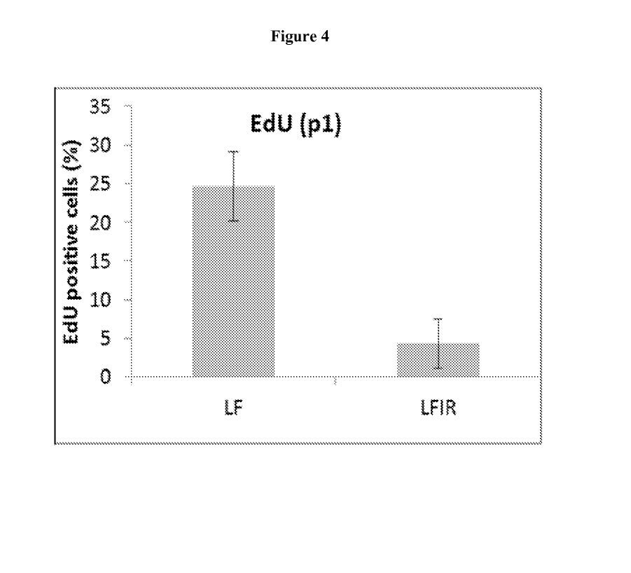

This Example provides a non-limiting example of analyzing cell proliferation to assist with detection of DSPCs. In this regard, and as shown by way of the data presented in FIG. 4, to determine whether lung mesenchymal cells proliferate when isolated from radiation treated mice, we measured EdU incorporation. Control (LF) cells and IR-treated (LFIR) cells were treated with Click-iT Edu in accordance to manufacturing instructions (Invitrogen). Proliferating cells (LF) stained positive for EdU incorporation (Red—top two image panels), while irradiation treated cells (LFIR) showed extremely minute amounts of EdU staining (bottom two image panels). Also see the bar graph. Therefore, lung mesenchymal cells isolated from untreated mice proliferate robustly in culture, whereas cells isolated from irradiation treated animals do not.

### EXAMPLE 5

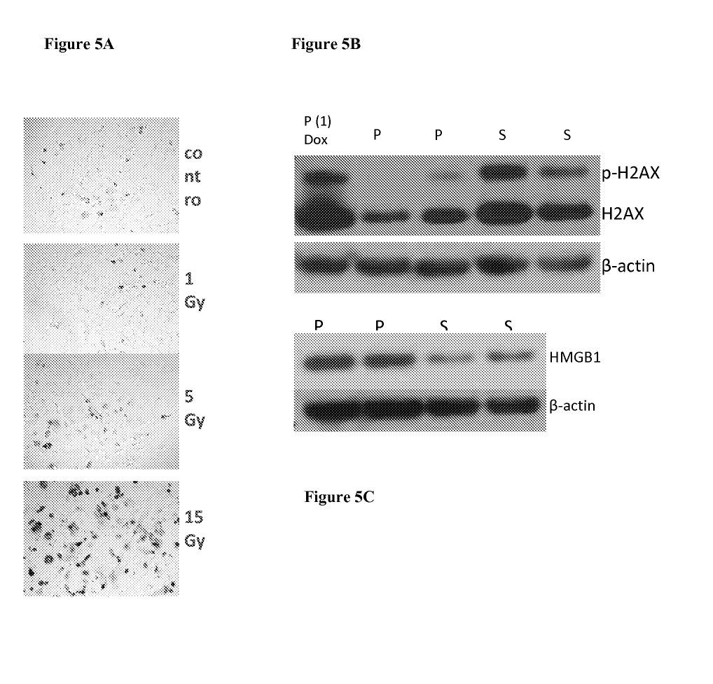

This Example provides a non-limiting example of analyzing conversion to a senescent phenotype. In this regard, as shown in FIGS. 5A-5C, to determine whether the lung mesenchymal cells isolated from IR-treated mice underwent senescence we assayed a number of established senescence associated markers. (FIG. 5A) Senescence associated beta-galactosidase activity was measured in mesenchymal lung cells following treatment with different doses of gamma-radiation. In this assay increase in beta-gal positive cells (blue cells) directly correlated with increasing dose of the radiation. (FIG. 5B) One of the markers of senescence is the presence of DNA damage in the cells. To determine whether the arrested cells have DNA damage, western immunoblotting was performed for phosphorylated gamma-H2AX protein. (S=senescent; P=proliferation; dox=doxorubicin). H2AX (H2A histone family member X) becomes phosphorylated under the conditions of double-stranded DNA break. (FIG. 5C) To determine whether the senescent cells have a decrease of HMGB1, we performed a western blot for anti-HMGB1 antibody. By analyzing a number of senescence associated markers in our mesenchymal lung cell model, we were able to detect the presence of these markers in our irradiation treated culture only, thus concluding that the state of arrest of irradiation treated sample can be defined as senescence.

### EXAMPLE 6

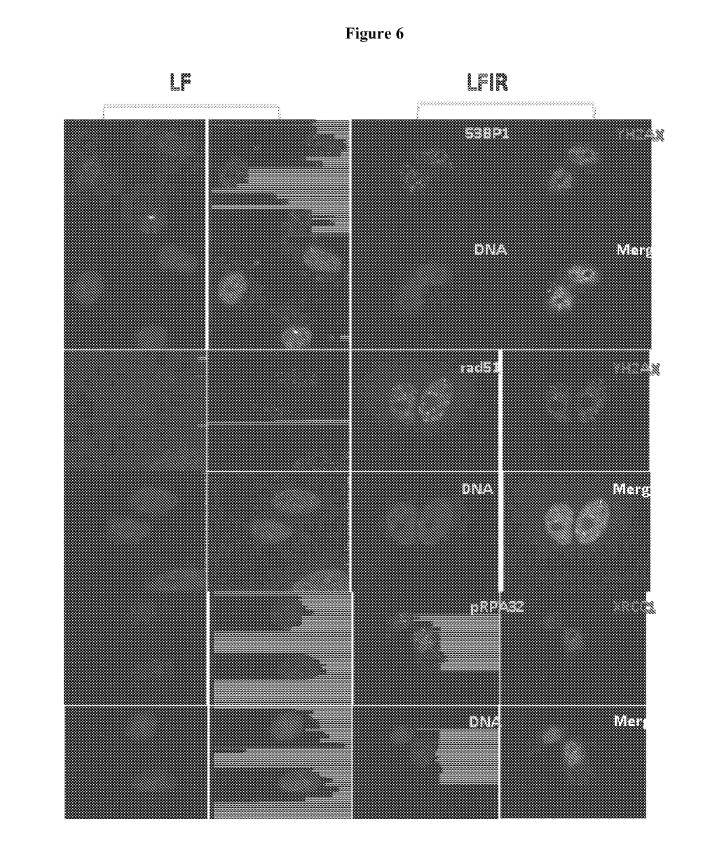

This Example provides a non-limiting example of analyzing DNA damage using an immunological approach. In this regard, as shown in FIG. 6, to further investigate the amount of DNA damage presented in the arrested cells, we performed immunohistochemistry with different DNA damaging markers that detect various damages (such as double strand and single strand breaks). We were able to establish that gamma-H2AX (H2AX (H2A histone family member X) becomes phosphorylated under the conditions of double-stranded DNA break) shows some level of foci in almost 100% of control (LF) cells. However, it is clearly induced in LF IR sample (both, number of foci/cell and foci size). 53BP1 (53BP1 binds to the central DNA-binding domain of p53 and is relocated to the sites of DNA strand breaks in response to DNA damage) had almost nothing in control cells but clear foci formation in LF IR in roughly 30% of cells. Some colocalization with gamma-H2AX, although much worse compared to Rad51. Rad51 (Rad51 redistribution to chromatin and nuclear foci formation is induced by double strand breaks) had almost nothing in control cells and clear foci in IR cells. Also, there is significant colocalization of Rad51 and gamma-H2AX foci. XRCC1 (XRCC1 is efficient in repairing single-strand breaks from ionizing radiation and alkylating agents) showed some level of foci in about 30% of control cells but it was clearly induced in LF IR (in more than 50%). Phosphor-RPA32 (Ser4/8) (pRPA32 binds to single-stranded DNA with high affinity. The 32 kDa subunit of RPA becomes hyper-phosphorylated in response to DNA damage and showed some level in control (about 30%) but it was clearly induced in IR (more than 50% of cells) treated cells. Based on the DNA damaging markers tested, more DNA damage was present in the senescent (irradiated) mesenchymal cells than the proliferating (untreated) cells.

### EXAMPLE 7

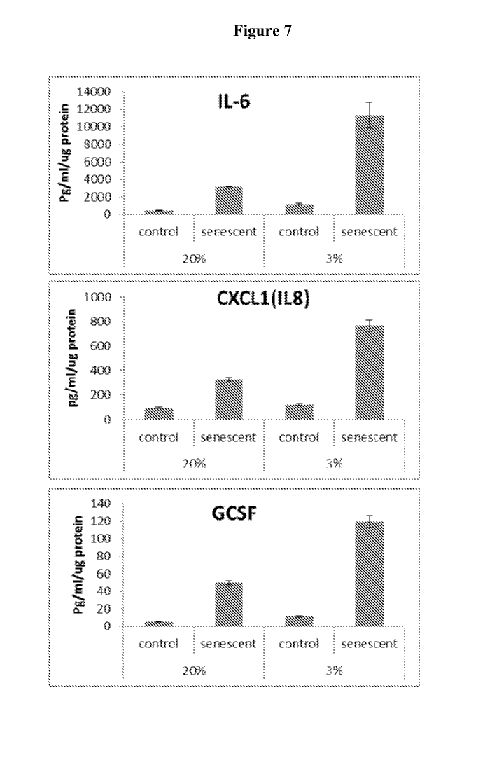

This Example provides a non-limiting demonstration of determining senescence-associated secretory phenotype (SASP). In this regard, FIG. 7 provides an analysis of three of the strongest induced inflammatory cytokines determined SASP, which are IL6, IL8 and GCSF. To obtain the data, proliferating or senescent cells were plated in 24-well format at 20,000 cells per well in 250 uL of DMEM medium. Cells were maintained either at 20% or 3% oxygen conditions. 72 hours later medium was collected and cytokines were assayed by flow cytometry. Cell number discrepancy was adjusted by normalizing the pg/ml cytokine value for cell number in each well. Senescent cells secreted higher amount of cytokines into the medium than proliferating cells.

### EXAMPLE 8

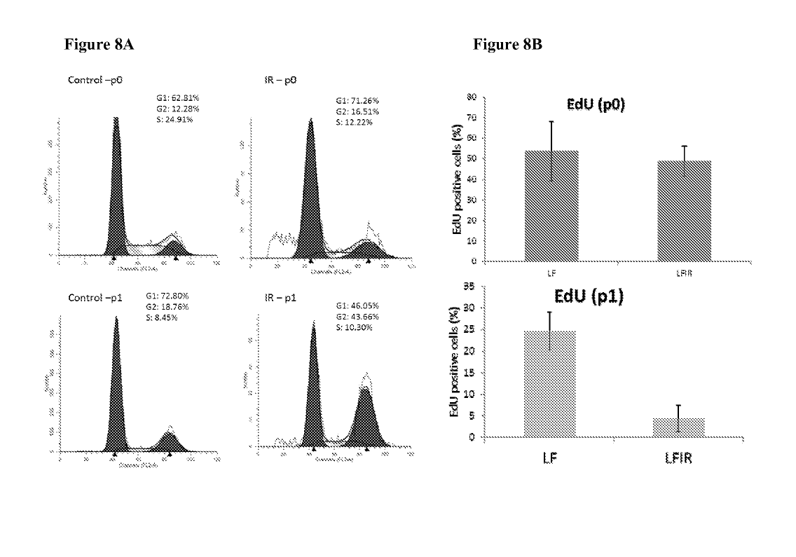

This Example provides a description of experiments performed to determine when cells isolated from the lung of irradiated mice plated in culture enter senescence. In this regard, FIGS. 8A-8B provide a cell cycle analysis. In particular, to determine when cells isolated from the lung of irradiated mice plated in culture enter senescence, we analyzed cell cycle distribution (FIG. 8A) and EdU incorporation (FIG. 8B). (FIG. 8A) Proliferating and senescent cells were stained with propidium iodide at passage 0 and passage 1. Cell cycle distribution revealed that at passage 0 most cells are in G1, while at passage 1 (when majority of irradiated cells are senescent) IR-treated cells there is more accumulation in G2 than in proliferating control. (FIG. 8B) To test proliferation capacity of radiation treated and untreated cells these cells were stained with EdU at passage 0 and passage 1. The difference in the EdU positive cells at passage 0 and passage 1 of IR-treated cells suggest that these cells do try to proliferate, however, they senesce at passage 1.

### EXAMPLE 9

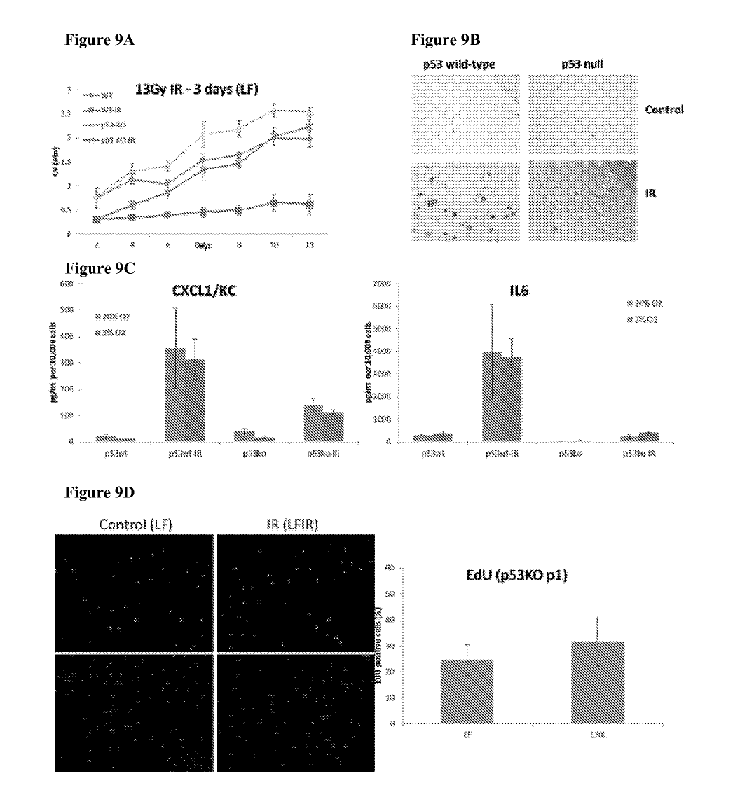

This Example provides an analysis of whether the senescence observed in irradiation treated cells is p53 dependent. As shown in FIGS. 9A-9D, we analyzed cells isolated from radiation treated and untreated p53 null mice to be compared with similarly treated p53 wild-type mice. (FIG. 9A) Cell doubling was determined by crystal violet over the period of 11 days. (FIG. 9B) To determine whether cells isolated from radiation treated p53-null mice are senescent, we stained IR-treated and untreated cells with beta-galactosidase. Only p53 wild-type treated with irradiation stain positive with senescence associated beta-galactosidase. (FIG. 9C) Senescence associated secretory phenotype was assayed in p53 wild-type and null cells isolated from IR-treated and untreated lung tissue. Cells isolated from p53-null mice regardless of the treatment do not secrete the same level of cytokines as irradiation treated p53-wild type cells. (FIG. 9D) To determine whether mesenchymal lung cells isolated from p53-null radiation treated and untreated mice continue to divide, EdU incorporation was measured. Regardless of the radiation the cells continue to divide.

### EXAMPLE 10

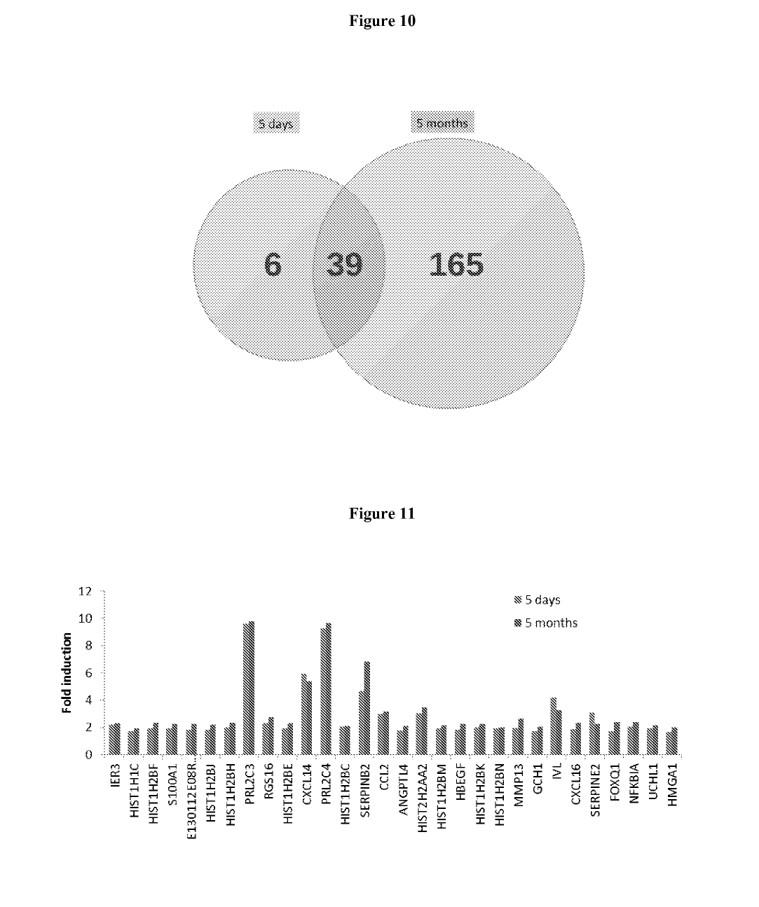

This Example provides an analysis of microarray data and identification of genes with common and opposite pattern of expression in primary lung cultures cells derived from irradiated mice after 5 days and 5 months. As shown in FIG. 10, we identified tgenes that belong to various families, such as pro-inflammatory genes, toll-like receptor, etc. The Venn diagram represents the number of genes that were upregulated in mouse lung fibroblasts isolated after 5 days post IR or 5 months post IR in comparison to untreated proliferating control. Microarray was performed in triplicates. Criteria for the data analysis were based on an average signal intensity to be greater than 500 and fold differences to be at least 1.5. Samples from group 2 and group 3 were compared with samples in group 1. Statistical analysis was performed using Microsoft Excel. The p-values were calculated using 2-sample t-test, assuming unequal variances. Values<0.05 were considered statistically significant.

### EXAMPLE 11

This Example provides an analysis of Illumina microarray-based analysis of gene expression in mouse lung derived fibroblasts in group that received in vivo gamma-radiation and were sacrificed either 5 days or 5 months after treatment. The results are summarized in FIG. 11.

### EXAMPLE 12

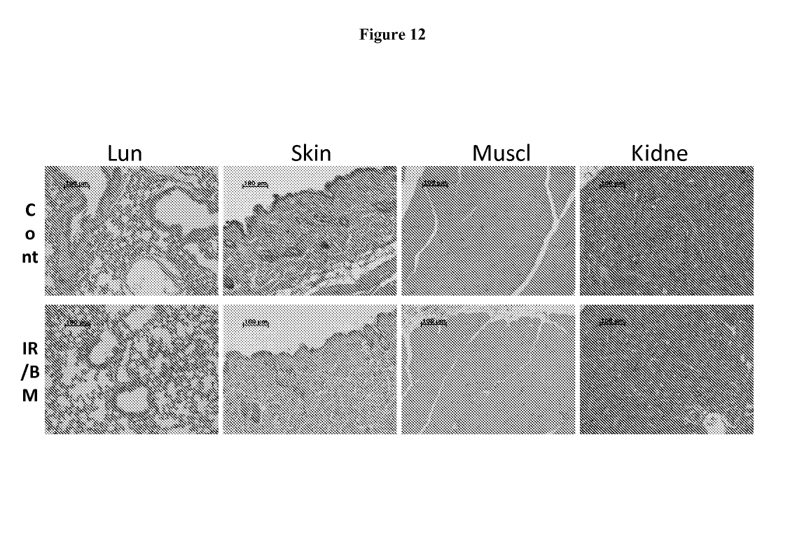

This Example provides a histochemical analysis of various tissues of untreated and IR treated animals, compared by H&E for any morphological differences. As shown in FIG. 12, C57BL/6 mice were treated with 11Gy of total body irradiation (TBI) and rescued by bone marrow transplantation (BMT). The tissues were collected and fixed three weeks after irradiation. The comparison between two groups revealed that there are no readily apparent differences between the tissues of the treated and untreated animals.

### EXAMPLE 13

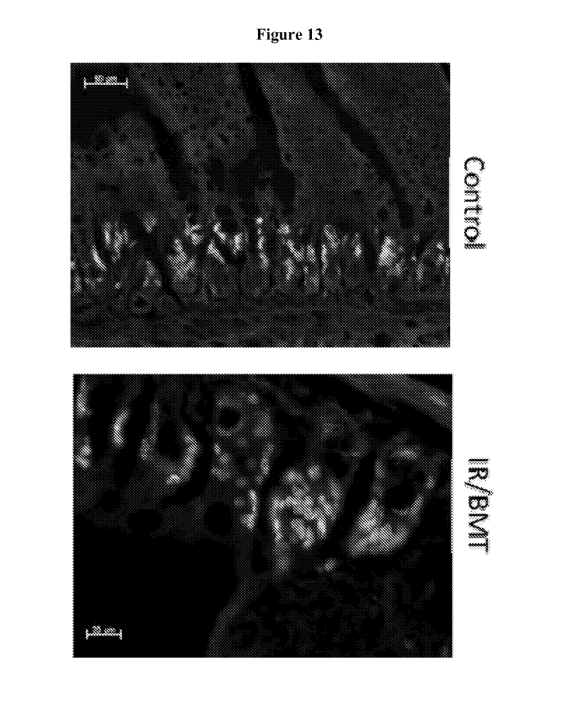

This Example provides a determination of whether there is a difference in the small intestine of intact (untreated) and IR-treated animals, EdU incorporation were measured. No difference between the two groups was detected, as shown in FIG. 13. In connection with this result, it will be recognized by those skilled in the art that the significance of the small intestine showing EdU incorporation to the same extent in irradiated as in non-irradiated mice is because the small intestine comprises rapidly proliferating tissue. In this regard, when mesenchymal cells from irradiated mice are forced to enter the cell cycle—they senesce and no longer can incorporate EdU, but the epithelial cells exhibit the same proliferate in both irradiated and non-irradiated animals. Thus, DSPC accumulation is tissue and cell specific, such that it is believed to be restricted to mesenchymal cells. Moreover, this result shows that in this in vivo model, after irradiation there are surprisingly no significant changes that occur in connection with a DNA damage response in the intestinal epithelial cells. However, for many years it has been assumed that irradiation alone is enough to cause premature aging in most if not all cell types. Thus, the present disclosure demonstrates that the mice that received lethal doses of irradiation and were subsequently rescued by bone marrow transplantation are histologically similar to their age-matched untreated control mice. Although the mice have a very high proportion of DSPCs among mesenchymal cells, the physiological effects of premature aging will only become evident when the DSPCs are converted into senescent cells, as would happen by consuming a high fat diet, or otherwise subjecting the cells to conditions that normally promote proliferation.

### EXAMPLE 14

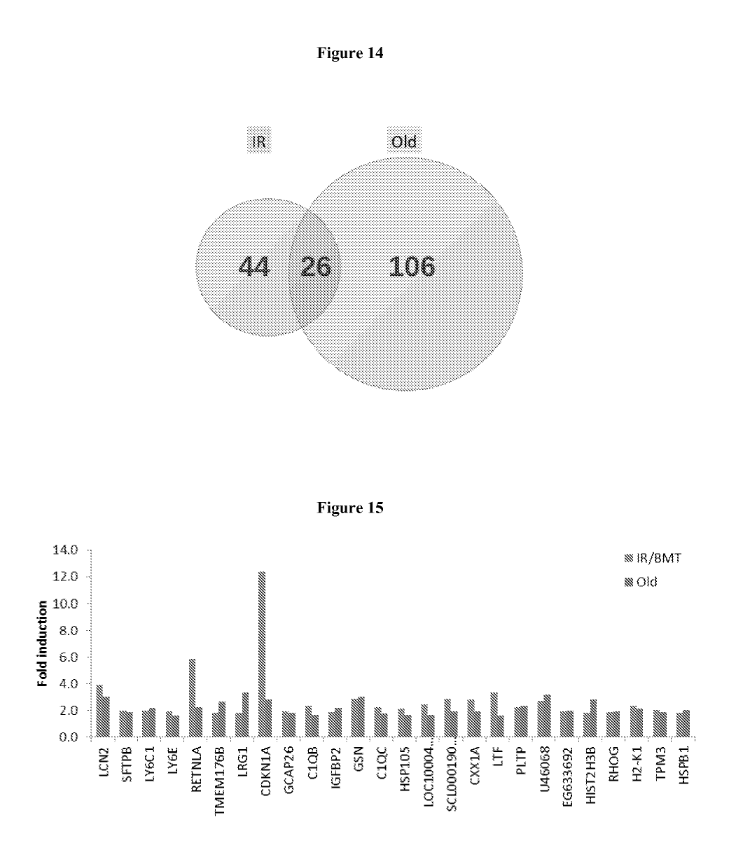

This example provides a description of data obtained from a microarray analysis performed on mouse lung tissue that varied in radiation treatment time and age. As shown in FIG. 14, for the mouse tissue array, three mouse groups were used, n=3 for each group. First group consisted of untreated young mice. Second group of mice received 11 Gy of gamma-radiation and were rescued by BMT and were sacrificed 3.5 weeks after irradiation. Last group of mice were untreated chronologically aged mice that were sacrificed at 1 year and nine months. We analyzed the RNAs level in lung tissue from IR and old mice and compared it to the RNA levels obtained from lung tissue of young untreated mice. 106 genes were upregulated in the lung tissue of the old mice, while only 44 genes where upregulated in the irradiated mice comparing to the control group. We have identified 26 genes, which are common for both groups.

### EXAMPLE 15

This Example demonstrates identification of genes upregulated in mouse lung tissue obtained from irradiated and naturally aged mice. The results are presented in FIG. 15.

### EXAMPLE 16

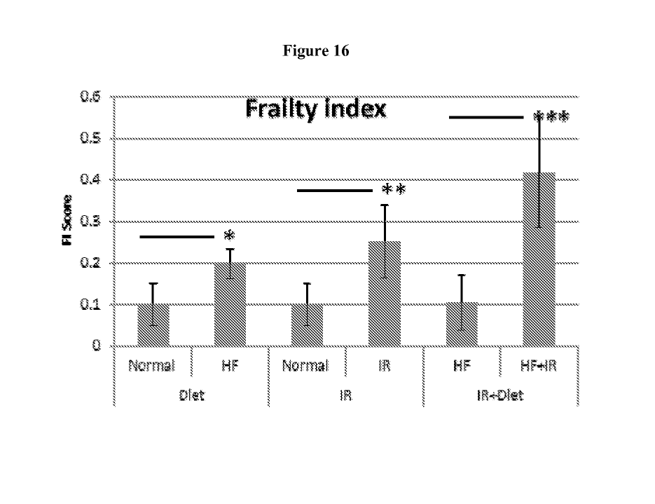

This Example provides a description of the determination of a Frailty Index. A graphical summary of FI is presented in FIG. 16. It was developed to assess a fit to frail range for the organisms of the same chronological age to address the notion that chronological age does not always reflect biologic age. Based on sixteen-item parameters (that include measurements of weight, grip strength, blood pressure, complete blood count, cytokine analysis) FI was calculated as a ratio of the total number of deficits measured and are assigned a score of FI between 0 (no deficits=fit) and 1 (all deficits present=frail). Therefore, higher FI indicates poorer health of an organism. In this regard, and as depicted in FIG. 16, a FI is provided as part of the current disclosure, and is useful for assessing a “fit” to “frail” range organisms of the same chronological age. As discussed above in the description of FIG. 16, based on a number of parameters, FI is calculated as a ratio of the total number of deficits measured, which is used to assign a score of FI between 0 (no deficits=fit) and 1 (all deficits present=frail). Thus, higher FI indicates a more pore health of an organism. To generate one illustrative example of determining FI, we estimated FI based on ten parameters including systolic and diastolic blood pressures, weight, grip strength, CXCL1 cytokine amount and CBC parameters. Four groups (n=7) was used to calculate FI, under four different conditions.

Group 1: Normal Diet; Intact Mice (Normal)

Group 2: High Fat Diet; Intact Mice (HF)

Group 3: Normal Diet; TBI IR Mice (IR)

Group 4: High Fat Diet; TBI IR Mice (HF-IR)

C57Bl/6 mice, with and without radiation (IR) were maintained either on a normal mouse diet (11% Fat) or a high-fat mouse diet (58%) (HF). To address how high fat diet changes FI, we compared the first two groups together. Group 1 and 3 were compared to determine the effect of IR on FI, and groups 2 and 4 were compared to determine how diet and irradiation together influences FI. There was statistical significance among the groups of mice (*p=0.005; **p=0.003; ***p=0.0008). Based on these parameters, we were able to determine that the animals that were placed on a high-fat diet after irradiation have a much higher FI, thus correlating with early aging and poorer health outcomes.

### EXAMPLE 17

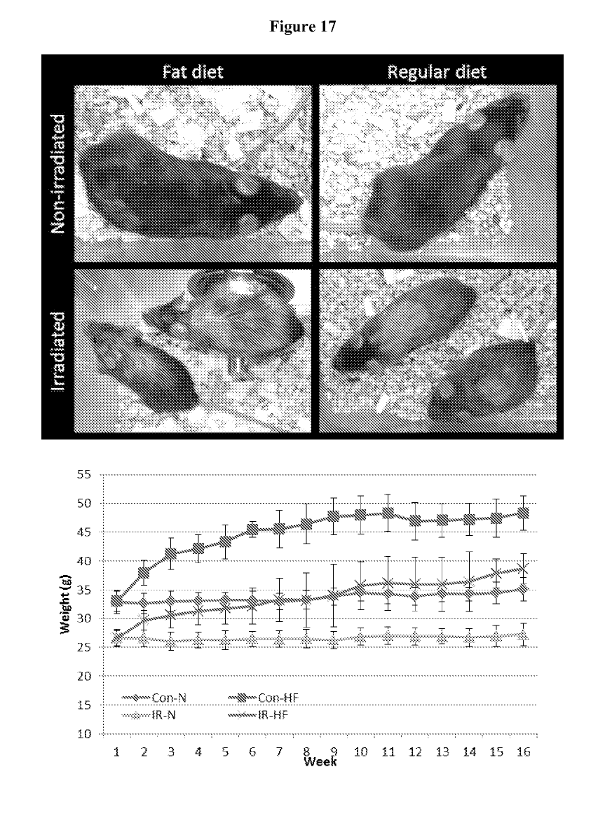

This Example provides a description of the effects on C57Bl/6 mice, with and without radiation (IR), and maintained either on a normal mouse diet (11% Fat) or a high-fat mouse diet (58%) (HF). Mouse weight was monitored once a week for 16 weeks. As shown in FIG. 17, intact, untreated animals increased their weight continuously over a prolonged period. However, the rate of weight gain in IR-treated and BMT-rescued animals was much slower than the untreated group.

### EXAMPLE 18

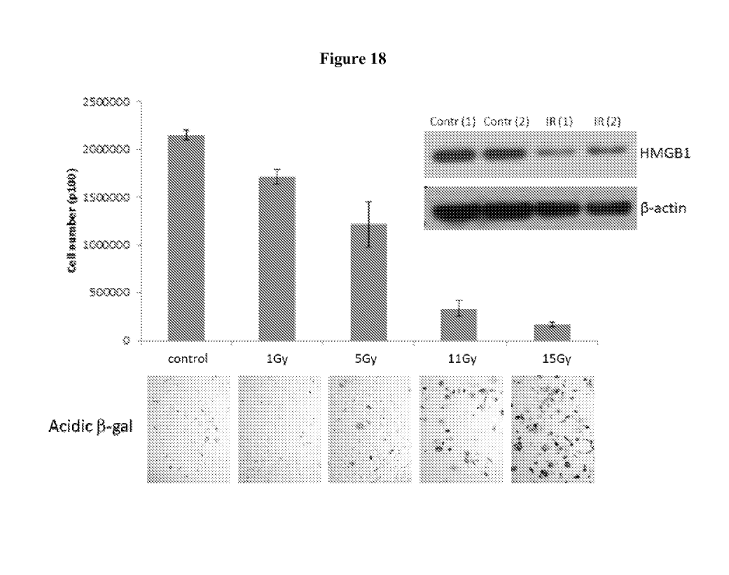

This Example provides a characterization of primary mouse lung fibroblasts that were isolated from C57Bl/6 mice 72 hours after various doses of total body of irradiation (0, 1, 5, 11, and 15Gy). As shown in FIG. 18, after a week in culture the number of fibroblasts was assessed via counting and senescence-associated β-galactosidase staining. We were able to determine by the amount of β-galactosidase/blue positive cells that there is a strong TBI dose dependence when DSPCs from in vivo are fully converted to senescent cells in vivo. Higher doses of irradiation correspond with greater damage received (but still not-recognized) in vivo, where upon plating in vitro and attempt to enter S-phase reveals this damage thus senescing the cells. To further characterize senescent phenotype of cells isolated from irradiated mice versus non-irradiated animals we performed Western immunoblotting for HMGB1 (high mobility group box 1). Levels of HMGB1 have been shown to be decreased in senescent cells. As seen in the Western blot, there is a striking loss of HMGB1 in cells isolated from irradiated animals (IR) versus proliferating cells, thus further confirming their senescent state.

### EXAMPLE 19

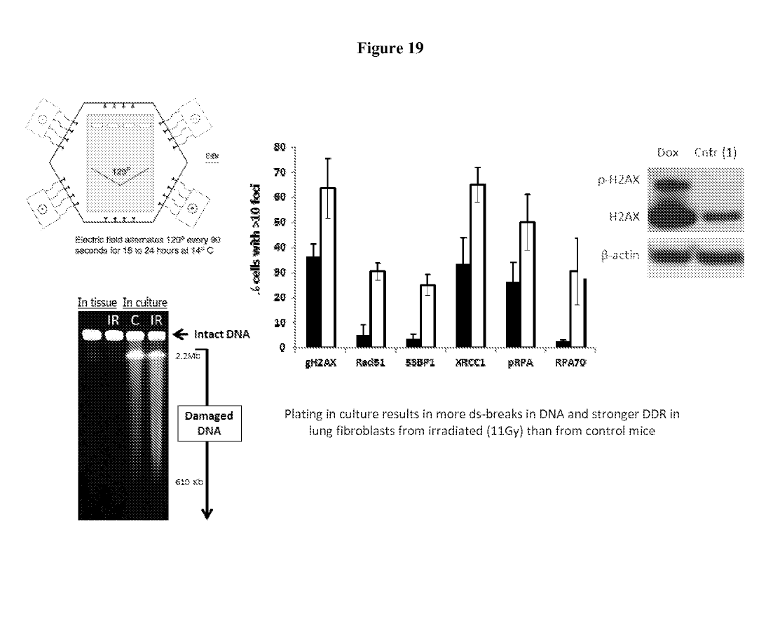

This Example demonstrates various parameters of DNA damage response (DDR) in senescent cells, such as greater levels of gH2AX, Rad51, 53BP1, XRCC1, pRPA, RPA70. These are the markers of double- or single breaks in DNA. In order to characterize mouse lung fibroblasts isolated from irradiated versus non-irradiated mice, it was considered important to compare DDR. To obtain the data summarized in FIG. 19, first, pulsed gel electrophoresis was performed using single lung cells suspension of irradiated and non-irradiated animals. We refer to these samples “in tissue” cells. The “in tissue” cells were compared to cells in culture. The in culture cells are mouse lung fibroblasts isolated from control and irradiated animals, which were analyzed seven days after plating, so that DSPCs would fully convert to SCs. Comparison of the two cell populations by pulsed-gel electrophoresis revealed that the cells in tissue do not acquire DNA damage response, presumably due to the fact they are non-dividing/quiescent cells. Cells in culture isolated from irradiated animals, however, show greater DNA damage response, which on the gel is represented as a smearing signal, than the cells from non-irradiated animals or cells in tissue. Moreover, LFIR (lung fibroblasts isolated from irradiated animals) and LF (lung fibroblasts from non-irradiated animals) were analyzed immunohistochemically (IHC) and by western immunoblotting for the presence of various nuclear markers of DDR. Using a large panel of DDR markers that detect single- and/or double stranded breaks we calculated percent of cells with greater than 10 positive foci for each of the protein listed. We established that cells isolated from irradiated animals have a greater number of cells with DDR foci than cells from non-irradiated animals.

### EXAMPLE 20

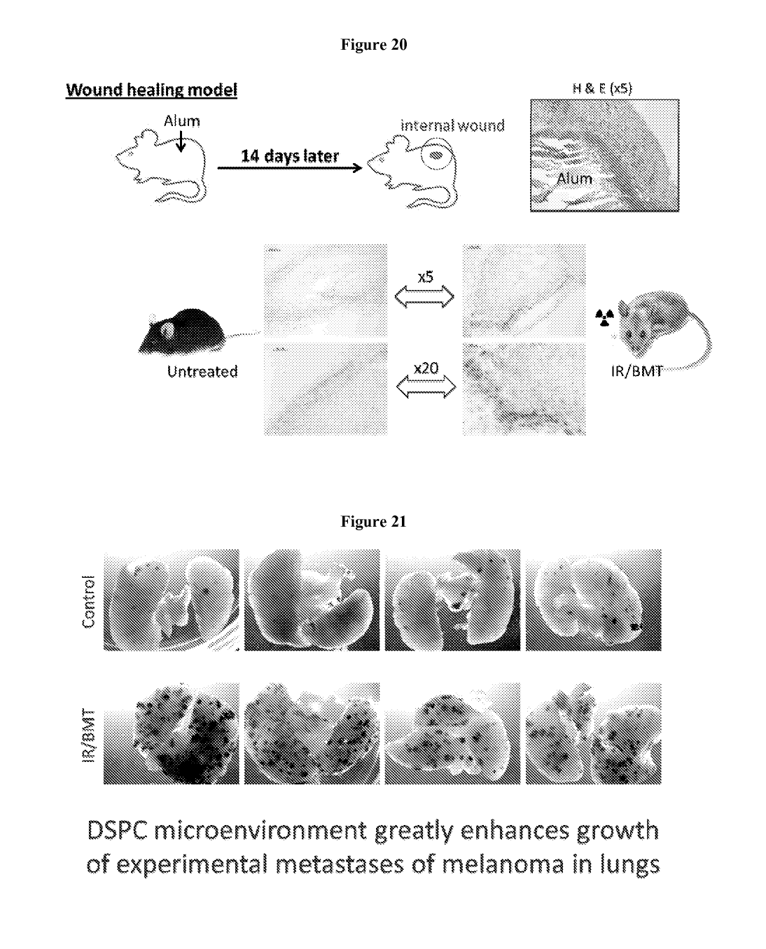

This Example demonstrates that DSPC conversion to fully senescent cells occurs in vitro during plating. In recent years, senescent cells have been implicated as critical components of wound healing, where during the process of wound healing/scar formation, senescent cells aid in recruiting necessary factors to expedite the process. In order to mimic these conditions in vivo, we tested whether an alum-based wound healing model would create the conditions to force the cells into division, thus forcing them to recognize DNA damage and senesce. We chose to use alum-based model, where alum is injected subcutaneously into irradiated and non-irradiated animals. As show in FIG. 20, after two weeks, fibrous capsules formed around the alum, which was then excised and analyzed for the presence of senescent cells using senescence-associated fl-galactosidase assay. The darker blue staining of the capsule excised from irradiated animals correlates with the discovery that when DSPCs are forced to proliferate they become senescent cells.

### EXAMPLE 21

It has been postulated that presence of senescent cells and the inflammatory factors that senescent cells secrete creates an environment that facilitates tumor growth. We tested whether we could convert DSPCs in vivo in mouse lung to SCs, and whether that would create conditions for greater tumor growth. In order to phenotypically reveal DSPC cells in vivo we utilized a B16 lung metastatic model. Irradiated or intact C57Bl/6 mice were injected via tail vein with B16 cells and two weeks later the lungs of these mice were isolated and formation of B16 metastasis was analyzed. B16 cells are pigmented mouse melanoma cells, which create black colonies in the lungs when grown in vivo. In this experiment, we were able to conclude that the DSPC microenvironment greatly enhances growth of experimental metastases of melanoma in lungs. The results are presented in FIG. 21.

TABLE 1
Illumina microarray analysis of transcripts upregulated 5 days and 5 months after gamma-
irradiation.

| | Fold | | | Signal intensity | | |
|---|---|---|---|---|---|---|
| Target ID | 5 days | 5 months | Target ID | Control | 5 days | 5 months |
| IER3 | 2.20 | 2.30 | IER3 | 3994.25 | 8806.25 | 9167.85 |
| HIST1H1C | 1.72 | 1.92 | HIST1H1C | 3521.1 | 6054.4 | 6756.6 |
| HIST1H2BF | 1.93 | 2.35 | HIST1H2BF | 2473.95 | 4769.4 | 5809.15 |
| S100A1 | 1.91 | 2.24 | S100A1 | 2138.9 | 4094.85 | 4780.95 |
| E130112E08RIK | 1.83 | 2.23 | E130112E08RIK | 2140.3 | 3921.6 | 4763.3 |
| HIST1H2BJ | 1.81 | 2.20 | HIST1H2BJ | 1725 | 3114.1 | 3789.7 |
| HIST1H2BH | 2.01 | 2.36 | HIST1H2BH | 1456.95 | 2922.65 | 3436.15 |
| PRL2C3 | 9.60 | 9.78 | PRL2C3 | 341.95 | 3281.6 | 3343.35 |
| RGS16 | 2.29 | 2.75 | RGS16 | 945.3 | 2168.2 | 2600.15 |
| HIST1H2BE | 1.92 | 2.28 | HIST1H2BE | 1114.35 | 2138.05 | 2541.85 |
| CXCL14 | 5.91 | 5.40 | CXCL14 | 459.3 | 2712.2 | 2482 |
| PRL2C4 | 9.28 | 9.64 | PRL2C4 | 256.85 | 2382.35 | 2476.8 |
| HIST1H2BC | 2.04 | 2.12 | HIST1H2BC | 958.25 | 1952.9 | 2029.75 |
| SERPINB2 | 4.68 | 6.84 | SERPINB2 | 295.1 | 1380.3 | 2017.45 |
| CCL2 | 3.00 | 3.19 | CCL2 | 620.05 | 1857.15 | 1975.85 |
| ANGPTL4 | 1.77 | 2.10 | ANGPTL4 | 920.4 | 1627.85 | 1929.15 |
| HIST2H2AA2 | 3.00 | 3.44 | HIST2H2AA2 | 506.85 | 1522.9 | 1743.15 |
| HIST1H2BM | 1.92 | 2.13 | HIST1H2BM | 592.55 | 1135.55 | 1262.6 |
| HBEGF | 1.81 | 2.26 | HBEGF | 526.4 | 953.8 | 1189.5 |
| HIST1H2BK | 2.01 | 2.23 | HIST1H2BK | 495.35 | 993.9 | 1103.55 |
| HIST1H2BN | 1.93 | 2.01 | HIST1H2BN | 526.1 | 1015.15 | 1055.25 |
| MMP13 | 1.96 | 2.64 | MMP13 | 383.35 | 749.75 | 1010.75 |
| GCH1 | 1.73 | 2.03 | GCH1 | 475.3 | 822 | 967.15 |
| IVL | 4.17 | 3.24 | IVL | 274.35 | 1144.3 | 890.15 |
| CXCL16 | 1.88 | 2.33 | CXCL16 | 368.55 | 691.7 | 857.25 |
| SERPINE2 | 3.09 | 2.24 | SERPINE2 | 377.1 | 1165.55 | 845.15 |
| FOXQ1 | 1.71 | 2.40 | FOXQ1 | 284.1 | 486.05 | 682.25 |
| NFKBIA | 2.05 | 2.40 | NFKBIA | 246.1 | 503.55 | 590.95 |
| UCHL1 | 1.93 | 2.17 | UCHL1 | 234.45 | 453.05 | 508.8 |
| HMGA1 | 1.67 | 1.99 | HMGA1 | 1097.70 | 1835.90 | 2181.45 |

TABLE 2
Illumina microarray analysis of transcripts upregulated 5 months after gamma-
irradiation.

| | Fold | | | Signal intensity | | |
|---|---|---|---|---|---|---|
| Target ID | 5 days | 5 months | Target ID | Control | 5 days | 5 months |
| PSAP | 1.15 | 2.15 | PSAP | 9617.20 | 11017.20 | 20704.15 |
| LOC100046120 | 1.12 | 2.44 | LOC100046120 | 6732.75 | 7567.15 | 16396.75 |
| LAPTM5 | 0.99 | 3.73 | LAPTM5 | 4242.15 | 4184.45 | 15843.65 |
| LGALS3 | 1.15 | 2.32 | LGALS3 | 6242.70 | 7179.10 | 14457.00 |
| SPP1 | 1.06 | 2.23 | SPP1 | 5607.35 | 5939.65 | 12506.65 |
| FCER1G | 0.58 | 5.05 | FCER1G | 2173.75 | 1262.90 | 10976.35 |
| SGK1 | 1.23 | 2.17 | SGK1 | 4822.85 | 5914.90 | 10449.15 |
| LOC100045864 | 1.62 | 4.59 | LOC100045864 | 2219.75 | 3590.00 | 10178.65 |
| CTSK | 0.82 | 3.78 | CTSK | 2179.15 | 1776.65 | 8242.35 |
| CCL9 | 0.64 | 3.61 | CCL9 | 2117.15 | 1353.40 | 7643.90 |
| CYBA | 1.06 | 2.18 | CYBA | 3295.30 | 3490.30 | 7175.15 |
| SH3BGRL3 | 1.36 | 2.40 | SH3BGRL3 | 2929.65 | 3970.00 | 7032.70 |
| CD9 | 0.86 | 2.23 | CD9 | 3120.05 | 2678.05 | 6960.25 |
| ALAS1 | 1.08 | 2.14 | ALAS1 | 3256.85 | 3531.10 | 6954.95 |
| CXCL4 | 0.61 | 4.82 | CXCL4 | 1380.95 | 847.10 | 6650.90 |
| CFP | 0.53 | 3.57 | CFP | 1852.80 | 979.00 | 6611.95 |
| LGMN | 0.85 | 2.18 | LGMN | 2780.10 | 2349.40 | 6065.05 |
| C1QB | 0.30 | 5.22 | C1QB | 1141.30 | 337.95 | 5957.65 |
| GPNMB | 1.16 | 3.14 | GPNMB | 1617.55 | 1873.70 | 5085.75 |
| RAB32 | 0.79 | 1.93 | RAB32 | 2413.70 | 1905.60 | 4660.70 |
| HGSNAT | 1.12 | 2.02 | HGSNAT | 2231.95 | 2492.60 | 4504.55 |
| H2-D1 | 1.52 | 5.43 | H2-D1 | 824.65 | 1253.00 | 4481.95 |
| CLEC4D | 1.02 | 7.58 | CLEC4D | 587.95 | 596.95 | 4457.25 |
| LYZS | 0.90 | 2.36 | LYZS | 1853.95 | 1659.45 | 4366.10 |
| FCGR4 | 1.01 | 5.50 | FCGR4 | 788.00 | 798.05 | 4337.15 |
| ARG1 | 0.30 | 2.71 | ARG1 | 1568.35 | 465.10 | 4249.65 |
| APOE | 0.62 | 8.48 | APOE | 492.50 | 303.70 | 4174.00 |
| LPL | 0.94 | 2.08 | LPL | 1910.55 | 1787.00 | 3974.60 |
| LIP1 | 1.07 | 4.79 | LIP1 | 827.35 | 881.20 | 3963.90 |
| RNF130 | 1.30 | 3.09 | RNF130 | 1260.00 | 1636.20 | 3892.40 |
| CCL6 | 0.81 | 3.56 | CCL6 | 1085.75 | 881.60 | 3860.65 |
| SIRPA | 0.78 | 3.45 | SIRPA | 1116.90 | 876.40 | 3849.10 |
| C1QC | 0.42 | 5.81 | C1QC | 637.35 | 265.30 | 3702.00 |
| COTL1 | 0.78 | 2.41 | COTL1 | 1525.00 | 1188.50 | 3680.60 |
| AADACL1 | 0.90 | 2.50 | AADACL1 | 1451.15 | 1311.50 | 3622.60 |
| PLA2G15 | 1.09 | 1.90 | PLA2G15 | 1890.65 | 2057.85 | 3589.35 |
| BTG1 | 1.39 | 2.22 | BTG1 | 1594.85 | 2210.50 | 3538.15 |
| WFDC2 | 1.43 | 4.75 | WFDC2 | 733.45 | 1045.85 | 3485.80 |
| CLEC4N | 1.34 | 7.44 | CLEC4N | 451.20 | 602.45 | 3354.85 |
| HEXA | 1.01 | 1.99 | HEXA | 1670.05 | 1690.25 | 3327.40 |
| BCL2A1B | 0.71 | 4.17 | BCL2A1B | 791.85 | 560.85 | 3303.65 |
| CD68 | 0.95 | 5.60 | CD68 | 580.70 | 550.80 | 3252.40 |
| SLC15A3 | 1.11 | 6.84 | SLC15A3 | 474.85 | 528.05 | 3247.70 |
| MAN2B1 | 1.10 | 2.56 | MAN2B1 | 1234.85 | 1357.45 | 3155.55 |
| GM2A | 1.07 | 2.23 | GM2A | 1356.10 | 1451.65 | 3030.10 |
| TPD52 | 1.13 | 2.06 | TPD52 | 1442.85 | 1625.75 | 2969.20 |
| TYROBP | 0.98 | 6.59 | TYROBP | 443.30 | 436.00 | 2919.35 |
| SDC3 | 1.19 | 2.55 | SDC3 | 1142.65 | 1358.80 | 2918.65 |
| OTTMUSG00000000971 | 1.04 | 4.14 | OTTMUSG00000000971 | 702.35 | 727.60 | 2908.35 |
| ALOX5AP | 0.48 | 4.13 | ALOX5AP | 694.10 | 330.50 | 2868.45 |
| MMP12 | 1.23 | 3.89 | MMP12 | 720.50 | 886.20 | 2802.25 |
| EG630499 | 1.56 | 4.23 | EG630499 | 652.20 | 1018.90 | 2759.20 |
| FCGR3 | 0.80 | 5.67 | FCGR3 | 478.65 | 381.85 | 2712.60 |
| LY6A | 1.38 | 3.71 | LY6A | 727.35 | 1004.10 | 2699.95 |
| MRC1 | 0.63 | 3.86 | MRC1 | 696.40 | 439.65 | 2686.90 |
| CLDN4 | 0.90 | 3.16 | CLDN4 | 834.30 | 753.70 | 2637.60 |
| DPP7 | 1.24 | 2.06 | DPP7 | 1279.65 | 1591.05 | 2637.25 |
| TREM2 | 0.72 | 7.18 | TREM2 | 365.60 | 265.05 | 2625.55 |
| MMP9 | 0.53 | 5.13 | MMP9 | 486.80 | 257.95 | 2497.35 |
| CYTH4 | 0.78 | 5.11 | CYTH4 | 486.30 | 378.05 | 2484.20 |
| CTSH | 0.99 | 2.99 | CTSH | 813.80 | 807.65 | 2436.60 |
| STXBP2 | 1.11 | 2.02 | STXBP2 | 1174.05 | 1308.75 | 2375.70 |
| CD52 | 0.66 | 7.01 | CD52 | 337.85 | 221.55 | 2368.25 |
| PRKCD | 1.11 | 1.90 | PRKCD | 1227.00 | 1365.30 | 2336.20 |
| ZFAND2A | 1.58 | 2.20 | ZFAND2A | 1058.40 | 1675.95 | 2323.45 |
| GLTP | 0.99 | 2.17 | GLTP | 1063.90 | 1053.30 | 2307.45 |
| LRRC8D | 1.48 | 2.10 | LRRC8D | 1072.50 | 1587.15 | 2253.35 |
| BLVRB | 1.54 | 2.42 | BLVRB | 931.75 | 1434.00 | 2253.05 |
| CHI3L3 | 1.06 | 4.21 | CHI3L3 | 528.60 | 558.50 | 2224.00 |
| CTSC | 0.64 | 2.59 | CTSC | 854.80 | 546.50 | 2215.85 |
| CTSZ | 1.22 | 3.10 | CTSZ | 707.25 | 861.65 | 2191.25 |
| PLEKHM2 | 1.03 | 2.18 | PLEKHM2 | 940.85 | 965.00 | 2050.40 |
| BCL2A1D | 0.63 | 4.28 | BCL2A1D | 472.95 | 295.65 | 2022.05 |
| NCKAP1L | 0.66 | 6.49 | NCKAP1L | 304.20 | 202.10 | 1973.65 |
| MS4A6D | 0.65 | 5.03 | MS4A6D | 386.25 | 250.20 | 1943.00 |
| ADFP | 1.34 | 2.85 | ADFP | 676.20 | 906.00 | 1926.25 |
| SLC40A1 | 0.83 | 4.31 | SLC40A1 | 432.20 | 357.65 | 1861.45 |
| LOC674135 | 1.68 | 4.82 | LOC674135 | 384.45 | 645.75 | 1851.70 |
| LRP12 | 0.90 | 2.14 | LRP12 | 847.30 | 760.15 | 1811.60 |
| AA467197 | 0.75 | 3.85 | AA467197 | 455.80 | 342.75 | 1753.35 |
| HSD3B7 | 1.02 | 2.07 | HSD3B7 | 844.10 | 857.15 | 1747.60 |
| LOC245892 | 0.85 | 2.42 | LOC245892 | 717.85 | 609.20 | 1739.50 |
| LHFPL2 | 1.22 | 2.13 | LHFPL2 | 805.60 | 986.65 | 1716.45 |
| P2RY6 | 0.81 | 4.87 | P2RY6 | 350.75 | 284.45 | 1708.10 |
| CD14 | 1.14 | 5.17 | CD14 | 328.95 | 375.95 | 1701.05 |
| SLC11A1 | 1.20 | 4.64 | SLC11A1 | 366.40 | 439.15 | 1699.55 |
| LY6E | 1.13 | 2.62 | LY6E | 642.35 | 725.45 | 1683.00 |
| MPEG1 | 0.90 | 4.27 | MPEG1 | 394.05 | 354.10 | 1680.70 |
| LOC100048461 | 0.81 | 3.31 | LOC100048461 | 504.30 | 410.80 | 1669.80 |
| SMPDL3A | 0.97 | 3.04 | SMPDL3A | 530.50 | 516.15 | 1612.85 |
| KRT7 | 1.07 | 3.54 | KRT7 | 450.40 | 480.60 | 1595.50 |
| SORT1 | 0.99 | 2.17 | SORT1 | 731.60 | 721.70 | 1587.50 |
| TSPAN14 | 0.87 | 2.10 | TSPAN14 | 752.70 | 652.25 | 1580.85 |
| GPR137B-PS | 0.82 | 2.10 | GPR137B-PS | 704.05 | 575.75 | 1481.50 |
| 2310016C08RIK | 1.46 | 3.39 | 2310016C08RIK | 435.25 | 635.90 | 1476.30 |
| CLECSF12 | 0.85 | 3.25 | CLECSF12 | 453.35 | 386.85 | 1473.10 |
| NGFB | 1.43 | 2.09 | NGFB | 685.85 | 982.45 | 1436.50 |
| MYO1F | 0.81 | 6.25 | MYO1F | 226.00 | 182.85 | 1413.20 |
| ARL11 | 0.81 | 6.41 | ARL11 | 219.25 | 176.95 | 1405.55 |
| CAPG | 1.20 | 2.48 | CAPG | 553.35 | 664.40 | 1372.15 |
| LMO2 | 0.59 | 4.12 | LMO2 | 319.50 | 187.15 | 1316.20 |
| CREG1 | 1.12 | 2.52 | CREG1 | 513.05 | 572.70 | 1292.75 |
| LOC676420 | 1.05 | 2.02 | LOC676420 | 637.55 | 669.30 | 1290.00 |
| KRT18 | 1.25 | 1.93 | KRT18 | 668.55 | 837.25 | 1288.15 |
| CHI3L4 | 1.19 | 4.74 | CHI3L4 | 269.20 | 321.20 | 1275.75 |
| CLEC7A | 1.06 | 5.35 | CLEC7A | 233.40 | 248.30 | 1249.75 |
| TGFB1 | 0.94 | 2.91 | TGFB1 | 427.30 | 400.50 | 1244.30 |
| CASP1 | 1.16 | 3.50 | CASP1 | 343.35 | 398.30 | 1202.30 |
| 2310007B03RIK | 0.92 | 2.49 | 2310007B03RIK | 481.30 | 440.65 | 1198.20 |
| RILPL2 | 1.06 | 3.38 | RILPL2 | 353.30 | 374.75 | 1192.80 |
| LCP1 | 0.54 | 3.55 | LCP1 | 316.40 | 171.85 | 1124.40 |
| TMEM86A | 0.96 | 2.27 | TMEM86A | 484.90 | 467.30 | 1103.10 |
| 1200002N14RIK | 1.15 | 2.33 | 1200002N14RIK | 458.10 | 525.25 | 1069.30 |
| 4933407C03RIK | 1.03 | 1.94 | 4933407C03RIK | 548.25 | 562.90 | 1066.15 |
| SGPL1 | 1.07 | 1.99 | SGPL1 | 515.10 | 548.75 | 1024.65 |
| TMEM205 | 1.38 | 2.15 | TMEM205 | 473.30 | 651.75 | 1017.75 |
| GPRC5A | 1.21 | 2.87 | GPRC5A | 350.25 | 423.60 | 1006.00 |
| JUNB | 1.09 | 2.11 | JUNB | 459.95 | 499.50 | 971.90 |
| TNFSF12-TNFSF13 | 0.84 | 2.68 | TNFSF12-TNFSF13 | 358.40 | 301.20 | 962.15 |
| SOX4 | 1.59 | 2.23 | SOX4 | 430.00 | 681.95 | 957.95 |
| 5033414K04RIK | 0.87 | 3.19 | 5033414K04RIK | 286.20 | 249.35 | 913.70 |
| SH3BP2 | 1.13 | 3.64 | SH3BP2 | 248.30 | 281.65 | 903.70 |
| TMEM51 | 1.32 | 2.22 | TMEM51 | 407.25 | 536.90 | 903.60 |
| 2310043N10RIK | 1.13 | 1.92 | 2310043N10RIK | 463.20 | 521.60 | 888.20 |
| ZRANB3 | 0.96 | 2.81 | ZRANB3 | 315.55 | 302.65 | 887.45 |
| MIB2 | 1.25 | 1.91 | MIB2 | 462.50 | 576.65 | 883.65 |
| ARHGEF3 | 1.42 | 2.21 | ARHGEF3 | 384.00 | 545.75 | 847.55 |
| CCL4 | 0.37 | 1.98 | CCL4 | 419.95 | 154.55 | 829.70 |
| CSF2RA | 0.84 | 3.73 | CSF2RA | 221.15 | 185.75 | 825.75 |
| RASSF5 | 1.17 | 2.30 | RASSF5 | 336.20 | 393.65 | 774.00 |
| SPINT1 | 1.24 | 2.73 | SPINT1 | 282.55 | 349.15 | 771.20 |
| PYGL | 0.95 | 1.91 | PYGL | 397.40 | 376.50 | 760.75 |
| RAI3 | 1.48 | 3.15 | RAI3 | 238.80 | 354.15 | 752.00 |
| RASSF3 | 1.34 | 2.00 | RASSF3 | 362.45 | 486.25 | 726.05 |
| TACSTD2 | 1.22 | 2.76 | TACSTD2 | 261.90 | 318.95 | 723.65 |
| GSTM1 | 1.39 | 2.79 | GSTM1 | 259.10 | 360.80 | 721.90 |
| MGLL | 1.41 | 2.63 | MGLL | 273.90 | 386.55 | 719.90 |
| SLC24A6 | 0.96 | 2.14 | SLC24A6 | 335.70 | 322.85 | 717.20 |
| CD93 | 0.81 | 2.70 | CD93 | 265.30 | 215.05 | 716.20 |
| GDF15 | 1.39 | 1.92 | GDF15 | 372.60 | 516.95 | 713.55 |
| TCIRG1 | 0.94 | 2.33 | TCIRG1 | 303.35 | 285.60 | 707.60 |
| SEMA4A | 1.09 | 2.88 | SEMA4A | 245.30 | 267.00 | 705.45 |
| IGK-C | 0.48 | 20.65 | IGK-C | 33.55 | 16.2 | 692.7 |
| KLF13 | 1.26 | 2.31 | KLF13 | 299.30 | 375.75 | 692.40 |
| CLN3 | 1.14 | 2.23 | CLN3 | 310.10 | 352.40 | 692.00 |
| 2510009E07RIK | 0.84 | 2.14 | 2510009E07RIK | 318.55 | 266.70 | 680.55 |
| DSCR1 | 1.57 | 1.96 | DSCR1 | 343.70 | 541.05 | 673.95 |
| PFKFB4 | 0.74 | 1.90 | PFKFB4 | 342.50 | 254.95 | 652.45 |
| EGR2 | 0.99 | 2.32 | EGR2 | 275.80 | 273.50 | 641.05 |
| RAB3D | 1.14 | 2.26 | RAB3D | 279.90 | 318.70 | 633.30 |
| MGC18837 | 1.38 | 2.36 | MGC18837 | 263.35 | 362.50 | 620.70 |
| KRT19 | 1.12 | 2.67 | KRT19 | 229.95 | 258.45 | 614.50 |
| TGFBI | 0.46 | 2.73 | TGFBI | 224.70 | 102.70 | 614.15 |
| ANXA11 | 1.20 | 2.12 | ANXA11 | 288.30 | 345.50 | 610.35 |
| KLF2 | 1.05 | 2.01 | KLF2 | 302.05 | 316.65 | 607.75 |
| SLC25A45 | 0.79 | 2.67 | SLC25A45 | 222.00 | 175.00 | 591.80 |
| FAM134B | 0.96 | 2.39 | FAM134B | 247.95 | 237.15 | 591.40 |
| IFNGR1 | 1.30 | 2.14 | IFNGR1 | 271.65 | 353.05 | 581.35 |
| MGST3 | 1.08 | 2.62 | MGST3 | 221.90 | 240.55 | 580.70 |
| HEBP1 | 1.06 | 2.05 | HEBP1 | 266.10 | 282.70 | 544.20 |
| SPHK2 | 1.22 | 1.96 | SPHK2 | 263.25 | 319.95 | 514.85 |
| TNFRSF21 | 0.75 | 2.02 | TNFRSF21 | 248.55 | 186.30 | 502.95 |
| RIN2 | 0.91 | 2.25 | RIN2 | 223.20 | 202.05 | 501.55 |
| CD82 | 1.53 | 2.12 | CD82 | 234.55 | 359.45 | 498.35 |
| ABHD12 | 1.26 | 2.20 | ABHD12 | 223.60 | 281.40 | 490.85 |

TABLE 3
Illumina microarray analysis of transcripts upregulated in IR and Old groups

| | Signal Intensity | | | | Fold | |
|---|---|---|---|---|---|---|
| Target ID | Young | IR/BMT | Old | Target ID | IR/BMT | Old |
| LCN2 | 2523.4 | 9980.7 | 7638.067 | LCN2 | 4.0 | 3.0 |
| SFTPB | 4663.4 | 9330.8 | 8815.767 | SFTPB | 2.0 | 1.9 |
| LY6C1 | 4401.333 | 8678.5 | 9574.167 | LY6C1 | 2.0 | 2.2 |
| LY6E | 3241.967 | 6223.5 | 5325.333 | LY6E | 1.9 | 1.6 |
| RETNLA | 842.8 | 4968.8 | 1904.867 | RETNLA | 5.9 | 2.3 |
| TMEM176B | 1188.967 | 2168.933 | 3165.6 | TMEM176B | 1.8 | 2.7 |
| LRG1 | 1061.8 | 1962.7 | 3563 | LRG1 | 1.8 | 3.4 |
| CDKN1A | 139.7 | 1730.267 | 392.8 | CDKN1A | 12.4 | 2.8 |
| GCAP26 | 771.1 | 1475.633 | 1403 | GCAP26 | 1.9 | 1.8 |
| C1QB | 594.0333 | 1399.867 | 1006.367 | C1QB | 2.4 | 1.7 |
| IGFBP2 | 688.1667 | 1283.667 | 1497.333 | IGFBP2 | 1.9 | 2.2 |
| GSN | 365.3333 | 1052.2 | 1104.333 | GSN | 2.9 | 3.0 |
| C1QC | 361.4 | 811.6667 | 651.1333 | C1QC | 2.2 | 1.8 |
| HSP105 | 368.3 | 785.2 | 621.1667 | HSP105 | 2.1 | 1.7 |
| LOC100048346 | 301.4667 | 748.3667 | 511.1333 | LOC100048346 | 2.5 | 1.7 |
| SCL0001905.1_3 | 236.6 | 680.1667 | 462.0333 | SCL0001905.1_3 | 2.9 | 2.0 |
| CXX1A | 234.4667 | 668.8333 | 450.7 | CXX1A | 2.9 | 1.9 |
| LTF | 197.1667 | 664.2 | 325.0333 | LTF | 3.4 | 1.6 |
| PLTP | 292.5 | 656.4 | 691.7333 | PLTP | 2.2 | 2.4 |
| U46068 | 236 | 638.8667 | 757.5 | U46068 | 2.7 | 3.2 |
| EG633692 | 287.4 | 561.1333 | 567.3667 | EG633692 | 2.0 | 2.0 |
| HIST2H3B | 305.5333 | 554.2 | 867.5 | HIST2H3B | 1.8 | 2.8 |
| RHOG | 290.4667 | 551.2667 | 568.9 | RHOG | 1.9 | 2.0 |
| H2-K1 | 227.6333 | 536.9667 | 483.9 | H2-K1 | 2.4 | 2.1 |
| TPM3 | 259.8333 | 529.7333 | 485.2667 | TPM3 | 2.0 | 1.9 |
| HSPB1 | 288.9 | 526.4333 | 587.4667 | HSPB1 | 1.8 | 2.0 |

TABLE 4
Illumina microarray analysis of transcripts upregulated in IR mouse lungs

| | Signal intensity | | | | Fold | |
|---|---|---|---|---|---|---|
| Target ID | Young | IR/BMT | Old | Target ID | IR/BMT | Old |
| HIST1H2AO | 3634.567 | 8400.167 | 2990.9 | HIST1H2AO | 2.3 | 0.8 |
| COL4A2 | 3027.867 | 5761.5 | 3981.367 | COL4A2 | 1.9 | 1.3 |
| RETNLA | 842.8 | 4968.8 | 1904.867 | RETNLA | 5.9 | 2.3 |
| H2-T23 | 2402.9 | 4753.3 | 2441.867 | H2-T23 | 2.0 | 1.0 |
| HIST1H2AD | 1906.167 | 4407.1 | 1553.833 | HIST1H2AD | 2.3 | 0.8 |
| IIGP2 | 1089.433 | 3777.267 | 1065.9 | IIGP2 | 3.5 | 1.0 |
| IGTP | 658.1 | 3301.5 | 758.5 | IGTP | 5.0 | 1.2 |
| LGALS3BP | 1107.767 | 3123.1 | 1753.8 | LGALS3BP | 2.8 | 1.6 |
| MMP2 | 1274.5 | 2924.1 | 1195 | MMP2 | 2.3 | 0.9 |
| FCGR4 | 1140.533 | 2611.533 | 1185.7 | FCGR4 | 2.3 | 1.0 |
| MMRN2 | 987.9667 | 2566.1 | 1117.833 | MMRN2 | 2.6 | 1.1 |
| SERPINA3N | 1210.633 | 2421.433 | 1794.8 | SERPINA3N | 2.0 | 1.5 |
| GBP2 | 986.4333 | 2283.767 | 824.3 | GBP2 | 2.3 | 0.8 |
| KNSL5 | 735.9667 | 2125.533 | 1376.667 | KNSL5 | 2.9 | 1.9 |
| PSMB8 | 640.2667 | 1742.367 | 998.7333 | PSMB8 | 2.7 | 1.6 |
| H2-Q5 | 881.2333 | 1704.733 | 1055.967 | H2-Q5 | 1.9 | 1.2 |
| HIST1H2AK | 511.2 | 1650.267 | 610.4333 | HIST1H2AK | 3.2 | 1.2 |
| HIST1H2AH | 484.8667 | 1576.9 | 552.7333 | HIST1H2AH | 3.3 | 1.1 |
| CD274 | 516.4667 | 1573.5 | 454.2333 | CD274 | 3.0 | 0.9 |
| CCL9 | 792.2667 | 1498.1 | 738.9 | CCL9 | 1.9 | 0.9 |
| PFN1 | 709.9667 | 1407.233 | 1285.3 | PFN1 | 2.0 | 1.8 |
| SERPINA3G | 486 | 1386.567 | 600.8 | SERPINA3G | 2.9 | 1.2 |
| DDAH1 | 655.4 | 1345.867 | 759.3667 | DDAH1 | 2.1 | 1.2 |
| GBP3 | 636.6 | 1332.167 | 378.8333 | GBP3 | 2.1 | 0.6 |
| A330102K04RIK | 290 | 1329.6 | 242.9333 | A330102K04RIK | 4.6 | 0.8 |
| EG667977 | 555.5 | 1245.667 | 928.2667 | EG667977 | 2.2 | 1.7 |
| PHLDA3 | 443.8 | 1163.3 | 611.1333 | PHLDA3 | 2.6 | 1.4 |
| TAP2 | 503.7 | 1030.867 | 806.6667 | TAP2 | 2.0 | 1.6 |
| CCL5 | 336.6 | 1008.9 | 531.2667 | CCL5 | 3.0 | 1.6 |
| NKG7 | 409.3 | 991.2333 | 248.7 | NKG7 | 2.4 | 0.6 |
| 5530400B01RIK | 458.2333 | 969.0333 | 537.3667 | 5530400B01RIK | 2.1 | 1.2 |
| H2-Q8 | 371.4667 | 946.5667 | 376.8 | H2-Q8 | 2.5 | 1.0 |
| PGLYRP1 | 451.0667 | 930.9333 | 676.9333 | PGLYRP1 | 2.1 | 1.5 |
| 0610037M15RIK | 322.4333 | 919.3333 | 396.9 | 0610037M15RIK | 2.9 | 1.2 |
| USP18 | 412.3 | 863.2 | 500.3333 | USP18 | 2.1 | 1.2 |
| WARS | 361.7 | 823.8667 | 344.0333 | WARS | 2.3 | 1.0 |
| SMPDL3B | 364.4667 | 801.7667 | 368.5667 | SMPDL3B | 2.2 | 1.0 |
| MCM5 | 306.4 | 786.3667 | 255.3 | MCM5 | 2.6 | 0.8 |
| H2-Q6 | 273.2667 | 711.2667 | 279.3667 | H2-Q6 | 2.6 | 1.0 |
| TINAGL1 | 341 | 663.0333 | 371.4333 | TINAGL1 | 1.9 | 1.1 |
| IRF9 | 323.2667 | 616.5667 | 311.9 | IRF9 | 1.9 | 1.0 |
| STAT1 | 304.3333 | 582.2 | 225.6333 | STAT1 | 1.9 | 0.7 |
| TIMP1 | 242.0667 | 557 | 243.2333 | TIMP1 | 2.3 | 1.0 |
| LOC100038882 | 190.8333 | 536.1667 | 138.5333 | LOC100038882 | 2.8 | 0.7 |

TABLE 5
Illumina microarray analysis of transcripts upregulated in Old mouse lungs

| | Signal intensity | | | | Fold | |
|---|---|---|---|---|---|---|
| Target ID | Young | IR/BMT | Old | Target ID | IR/BMT | Old |
| IGK-C | 5984.067 | 4135.767 | 16541.3 | IGK-C | 0.7 | 2.8 |
| LOC100047628 | 2120.1 | 1520.767 | 7058.133 | LOC100047628 | 0.7 | 3.3 |
| EAR4 | 3227.5 | 2006.3 | 6441.633 | EAR4 | 0.6 | 2.0 |
| CCL21A | 3086.6 | 3969 | 6113.233 | CCL21A | 1.3 | 2.0 |
| IGH-VJ558 | 2012.367 | 1246.533 | 5483.967 | IGH-VJ558 | 0.6 | 2.7 |
| CHI3L3 | 2113.9 | 911.9333 | 4394.667 | CHI3L3 | 0.4 | 2.1 |
| EG622339 | 2067.667 | 3151.233 | 4314.933 | EG622339 | 1.5 | 2.1 |
| CHIA | 1672.067 | 1662.867 | 4014.9 | CHIA | 1.0 | 2.4 |
| LOC100041504 | 1772.767 | 2160.1 | 3924.233 | LOC100041504 | 1.2 | 2.2 |
| CCL21C | 1418.933 | 1927.167 | 3551.033 | CCL21C | 1.4 | 2.5 |
| CHI3L4 | 1579.333 | 659.0667 | 3321.333 | CHI3L4 | 0.4 | 2.1 |
| SLPI | 1461.667 | 2388.767 | 3223.867 | SLPI | 1.6 | 2.2 |
| TMEM109 | 1675.933 | 1978.133 | 3213.233 | TMEM109 | 1.2 | 1.9 |
| MTDNA_ND2 | 1085.9 | 828.3 | 3203.9 | MTDNA_ND2 | 0.8 | 3.0 |
| IGH-6 | 1030 | 923.5333 | 3184.667 | IGH-6 | 0.9 | 3.1 |
| MT-CO2 | 1155.033 | 1018.067 | 3038.767 | MT-CO2 | 0.9 | 2.6 |
| ALDH1A1 | 1289.833 | 1143.367 | 2798.467 | ALDH1A1 | 0.9 | 2.2 |
| LOC383308 | 1370.967 | 1186.733 | 2772.567 | LOC383308 | 0.9 | 2.0 |
| RPL18A | 1001.767 | 666.4333 | 2752.367 | RPL18A | 0.7 | 2.7 |
| ACTA2 | 1004.067 | 1482.6 | 2678.267 | ACTA2 | 1.5 | 2.7 |
| TACSTD2 | 929.9 | 1032.867 | 2583.433 | TACSTD2 | 1.1 | 2.8 |
| EDN1 | 907.7 | 1189.2 | 2558.933 | EDN1 | 1.3 | 2.8 |
| LOC386067 | 923.8 | 1098.933 | 2557.567 | LOC386067 | 1.2 | 2.8 |
| DAZAP2 | 1292.767 | 1343 | 2545.333 | DAZAP2 | 1.0 | 2.0 |
| MT-ATP6 | 1301 | 839.7 | 2462.8 | MT-ATP6 | 0.6 | 1.9 |
| EG637748 | 1195.867 | 854.6667 | 2309.167 | EG637748 | 0.7 | 1.9 |
| CAR4 | 1086.2 | 1682.533 | 2228.367 | CAR4 | 1.5 | 2.1 |
| LOC381774 | 407.9333 | 327.2333 | 1992.567 | LOC381774 | 0.8 | 4.9 |
| EG433923 | 993.9333 | 1562.033 | 1962.633 | EG433923 | 1.6 | 2.0 |
| GSTM1 | 855.5333 | 914.2333 | 1706.033 | GSTM1 | 1.1 | 2.0 |
| H2-AA | 532.5 | 921.1333 | 1689.633 | H2-AA | 1.7 | 3.2 |
| PRDX5 | 843.2333 | 815.2333 | 1588.133 | PRDX5 | 1.0 | 1.9 |
| SPP1 | 195.7333 | 304.4 | 1507.567 | SPP1 | 1.6 | 7.7 |
| IGFBP2 | 688.1667 | 1283.667 | 1497.333 | IGFBP2 | 1.9 | 2.2 |
| LYVE1 | 678.9667 | 1041.8 | 1479.3 | LYVE1 | 1.5 | 2.2 |
| 1600029I14RIK | 716.8333 | 721.2333 | 1429.6 | 1600029I14RIK | 1.0 | 2.0 |
| ACOT1 | 742.5333 | 788.4667 | 1427.667 | ACOT1 | 1.1 | 1.9 |
| LOC100047162 | 306.8 | 285.4333 | 1426.533 | LOC100047162 | 0.9 | 4.6 |
| IGK-V5 | 281.1 | 217.6 | 1376.5 | IGK-V5 | 0.8 | 4.9 |
| NPC2 | 565.0667 | 598.0333 | 1357.533 | NPC2 | 1.1 | 2.4 |
| RETNLG | 628.2333 | 736.4667 | 1302.067 | RETNLG | 1.2 | 2.1 |
| LOC277856 | 611.3 | 636.1667 | 1301.133 | LOC277856 | 1.0 | 2.1 |
| PODXL | 604.5 | 848.1 | 1278.533 | PODXL | 1.4 | 2.1 |
| LOC433943 | 452.3 | 569.0667 | 1216.133 | LOC433943 | 1.3 | 2.7 |
| BC024561 | 565.2333 | 901.0667 | 1196.2 | BC024561 | 1.6 | 2.1 |
| LOC383010 | 532.1333 | 784.9333 | 1176.767 | LOC383010 | 1.5 | 2.2 |
| DYNLT3 | 578.1333 | 520.2 | 1164.133 | DYNLT3 | 0.9 | 2.0 |
| TUBA1A | 494.5 | 774.4333 | 1156.767 | TUBA1A | 1.6 | 2.3 |
| SOX18 | 602.7333 | 1020 | 1145.733 | SOX18 | 1.7 | 1.9 |
| NME5 | 423.8333 | 495.6667 | 1108.9 | NME5 | 1.2 | 2.6 |
| GSN | 365.3333 | 1052.2 | 1104.333 | GSN | 2.9 | 3.0 |
| CYP2A5 | 424.0333 | 318 | 1098.2 | CYP2A5 | 0.7 | 2.6 |
| HIST1H2BC | 481.7 | 600.7333 | 1077.833 | HIST1H2BC | 1.2 | 2.2 |
| CTSK | 516.6 | 741.2333 | 1060.433 | CTSK | 1.4 | 2.1 |
| DMKN | 515.7333 | 513.7333 | 1040.767 | DMKN | 1.0 | 2.0 |
| D14ERTD449E | 422.2333 | 346.0333 | 1020.767 | D14ERTD449E | 0.8 | 2.4 |
| CXCL17 | 482.8667 | 783.7 | 1009.467 | CXCL17 | 1.6 | 2.1 |
| KRT19 | 493.9667 | 621.5 | 983.0667 | KRT19 | 1.3 | 2.0 |
| FMO3 | 374.3 | 248 | 956.0667 | FMO3 | 0.7 | 2.6 |
| ALDH3A1 | 441.5333 | 520.6333 | 947.5 | ALDH3A1 | 1.2 | 2.1 |
| NRN1 | 442.1333 | 378.5 | 924.7667 | NRN1 | 0.9 | 2.1 |
| MYL9 | 449.0667 | 462.5 | 924.0667 | MYL9 | 1.0 | 2.1 |
| BC048546 | 312.1333 | 512.6333 | 920.7333 | BC048546 | 1.6 | 2.9 |
| GSTT3 | 452.9 | 425.7333 | 910.3333 | GSTT3 | 0.9 | 2.0 |
| SLC25A3 | 366.6333 | 538.4667 | 870.3667 | SLC25A3 | 1.5 | 2.4 |
| HIST2H3B | 305.5333 | 554.2 | 867.5 | HIST2H3B | 1.8 | 2.8 |
| ACTC1 | 283.5 | 387.2 | 856.5 | ACTC1 | 1.4 | 3.0 |
| ARPC2 | 397.8667 | 448.2333 | 820.7333 | ARPC2 | 1.1 | 2.1 |
| 1700009P17RIK | 369.5333 | 369.1333 | 808.5 | 1700009P17RIK | 1.0 | 2.2 |
| SOX7 | 359 | 573.1 | 808.4 | SOX7 | 1.6 | 2.3 |
| 4933427G23RIK | 413.2 | 318.7 | 803.9333 | 4933427G23RIK | 0.8 | 1.9 |
| LOC100048480 | 249.0667 | 251.9 | 791.7667 | LOC100048480 | 1.0 | 3.2 |
| ACAA2 | 391.5 | 519.8333 | 784.8 | ACAA2 | 1.3 | 2.0 |
| LOC637227 | 189.3 | 219.5 | 769.1333 | LOC637227 | 1.2 | 4.1 |
| U46068 | 236 | 638.8667 | 757.5 | U46068 | 2.7 | 3.2 |
| GAL | 367.8333 | 230 | 757.2333 | GAL | 0.6 | 2.1 |
| ARL8A | 354.6333 | 439.8 | 756.5 | ARL8A | 1.2 | 2.1 |
| ACTG2 | 244.8 | 316.8667 | 716.1333 | ACTG2 | 1.3 | 2.9 |
| EMB | 370.1333 | 404.9 | 700.9667 | EMB | 1.1 | 1.9 |
| PLTP | 292.5 | 656.4 | 691.7333 | PLTP | 2.2 | 2.4 |
| 1700001C02RIK | 303.7 | 364.1667 | 635.0667 | 1700001C02RIK | 1.2 | 2.1 |
| BCAP31 | 333.8667 | 419.6667 | 630.7 | BCAP31 | 1.3 | 1.9 |
| POLR2G | 263.0333 | 269 | 614.6667 | POLR2G | 1.0 | 2.3 |
| CCT8 | 320.1 | 327 | 613.2 | CCT8 | 1.0 | 1.9 |
| LOC381365 | 253.9667 | 231.9 | 602.6 | LOC381365 | 0.9 | 2.4 |
| HSPB1 | 288.9 | 526.4333 | 587.4667 | HSPB1 | 1.8 | 2.0 |
| 1700007G11RIK | 281.0333 | 264.5333 | 577.2 | 1700007G11RIK | 0.9 | 2.1 |
| 1110049B09RIK | 304.6667 | 371.2333 | 573.9667 | 1110049B09RIK | 1.2 | 1.9 |
| HMGCS2 | 288.1333 | 269.1333 | 573.1333 | HMGCS2 | 0.9 | 2.0 |
| LOC381649 | 206.4 | 236.8 | 569.9 | LOC381649 | 1.1 | 2.8 |
| RHOG | 290.4667 | 551.2667 | 568.9 | RHOG | 1.9 | 2.0 |
| EG633692 | 287.4 | 561.1333 | 567.3667 | EG633692 | 2.0 | 2.0 |
| SLC6A2 | 284.3333 | 331.8 | 562.3667 | SLC6A2 | 1.2 | 2.0 |
| TCN2 | 258.3333 | 351.6333 | 559.9333 | TCN2 | 1.4 | 2.2 |
| 1700027A23RIK | 229.5 | 196.5 | 542.9333 | 1700027A23RIK | 0.9 | 2.4 |
| MGST2 | 256.9 | 159.4 | 534.8333 | MGST2 | 0.6 | 2.1 |
| HADHB | 199.6667 | 223.3333 | 523.4667 | HADHB | 1.1 | 2.6 |
| PLUNC | 119.1333 | 87.3 | 515.2 | PLUNC | 0.7 | 4.3 |
| ABP1 | 193.7333 | 199.3333 | 500.4 | ABP1 | 1.0 | 2.6 |
| CXCL4 | 201.2667 | 138.8 | 498.7667 | CXCL4 | 0.7 | 2.5 |
| H2-K1 | 227.6333 | 536.9667 | 483.9 | H2-K1 | 2.4 | 2.1 |
| EAR10 | 132.4 | 65.43333 | 472.7 | EAR10 | 0.5 | 3.6 |
| LOC100042773 | 178.7 | 248.0667 | 471.2 | LOC100042773 | 1.4 | 2.6 |
| SCL0001905.1_3 | 236.6 | 680.1667 | 462.0333 | SCL0001905.1_3 | 2.9 | 2.0 |
| EAR12 | 119.3333 | 59.53333 | 460.0667 | EAR12 | 0.5 | 3.9 |
| WASF2 | 231.4333 | 306.8333 | 458.2333 | WASF2 | 1.3 | 2.0 |

While the invention has been described through specific embodiments, routine modifications will be apparent to those skilled in the art and such modifications are intended to be within the scope of the present invention.

What is claimed is:

1. A method for determining an amount of dormant senescence prone cells, the method comprising:
a) obtaining a biological sample comprising mesenchymal cells from a human individual or non-human animal;
b) placing the biological sample under conditions which promote cell proliferation, and subsequently measuring indicia of DNA damage response in the mesenchymal cells to obtain a measurement of the amount of dormant senescence prone cells in the biological sample, wherein the DNA damage response is in the dormant senescent prone cells, and wherein the amount of dormant senescent prone cells is a proportion of the mesenchymal cells.

2. The method of claim 1, wherein the indicia of DNA damage response is compared to a reference to obtain a measurement of the degree of genotoxic stress the human individual or non-human animal from which the biological sample was obtained experienced during its lifetime before the sample was obtained.

3. The method of claim 2, wherein the genotoxic stress comprised exposure to ionizing radiation, or having been treated with a chemotherapeutic drug which damages DNA, or a combination of the ionizing radiation and exposure to the chemotherapeutic drug.

4. The method of claim 1, wherein the placing the biological sample under conditions which promote cell proliferation is performed ex vivo and wherein the biological sample comprises a tissue sample, or wherein the placing the biological sample under conditions which promote cell proliferation is performed by plating cells from the biological sample in vitro.

5. The method of claim 1, wherein the biological sample comprises a first biological sample, and wherein the reference comprises a second biological sample comprising mesenchymal cells from the individual, the method comprising:
a) in the first biological sample, measuring indicia of DNA damage response in the mesenchymal cells after the placing them in the conditions promoting proliferation, and allowing a period of time to pass during which proliferation takes place in cells that do not exhibit the DNA damage response; and
b) in the second biological sample, measuring indicia of the DNA damage response before promotion of proliferation (pre-proliferation promotion cells);
wherein an increase in the indicia of the DNA damage response in the cells of a) relative to the indicia of DNA damage response in the pre-proliferation cells of b) indicates the biological sample comprised dormant senescent prone cells, and wherein the amount of increase in the indicia comprises a measurement of the degree of genotoxic stress the human individual or non-human animal experienced during its lifetime before the sample was obtained.

6. The method of claim 5, wherein the first and second biological samples are obtained from dividing a single sample into the first and second biological samples.

7. The method of claim 1, wherein the indicia of DNA damage response comprises an indicator of DNA damage response selected from the group consisting of: phosphorylation of a histone, nuclear foci comprising 53BP1, nuclear foci comprising Rad51, phosphorylation of RPA32, or secretion of a cytokine associated with senescence-associated secretory phenotype (SASP), wherein the cytokine is selected from IL6, IL8 and GCSF, and combinations thereof.

8. The method of claim 7, wherein the phosphorylation of the histone or the phosphorylation of RPA32, or the nuclear foci comprising 53BP1, or RPA32, or a combination thereof, is determined using an immunological assay.

9. The method of claim 7, wherein the phosphorylation is of H2A histone.

10. The method of claim 1, wherein the biological sample comprises a sample of tissue from the individual.

11. The method of claim 1, wherein the biological sample is determined to comprise dormant senescent prone cells, the method further comprising recommending to the individual to avoid weight gain.

12. The method of claim 1, wherein the biological sample is determined to comprise dormant senescent prone cells, the method further comprising recommending to the individual to avoid exposure to ionizing radiation.

13. The method of claim 1, wherein the biological sample is determined to comprise dormant senescent prone cells, the method further comprising determining the degree of the indicia of the DNA damage response and estimating an amount of one or more DNA damaging agents received by the individual before the biological sample was obtained.

14. The method of claim 13, wherein the DNA damaging agent is selected from ionizing radiation and drugs that inhibit cell division.

15. The method of claim 1, wherein the biological sample is determined to comprise dormant senescent prone cells, the method further comprising assigning a biological age to the individual, wherein the biological age is greater than the chronological age of the individual.

16. The method of claim 1, wherein the biological sample is determined to comprise dormant senescent prone cells, the method further comprising administering to the individual an agent that selectively kills dormant senescent cells.
